(19) United States
(12) Patent Application Publication
(10) Pub. No.: US 2024/0261270 A1
(43) Pub. Date: Aug. 8, 2024

(54) METHODS OF TREATING GASTROINTESTINAL STROMAL TUMORS

(71) Applicant: Deciphera Pharmaceuticals, LLC, Waltham, MA (US)

(72) Inventors: Jing Wang, Waltham, MA (US); Xiaoyan Li, Lexington, MA (US)

(21) Appl. No.: 18/464,519

(22) Filed: Sep. 11, 2023

Related U.S. Application Data
(63) Continuation of application No. 17/938,353, filed on Oct. 6, 2022, now Pat. No. 11,779,572.
(60) Provisional application No. 63/403,444, filed on Sep. 2, 2022.

(51) Int. Cl. A61K 31/4375; A61P 35/00

(52) U.S. Cl. CPC A61K 31/4375; A61P 35/00

(57) ABSTRACT

The present disclosure relates to methods of treating gastrointestinal stromal tumors to a subject in need thereof, comprising administering to the subject a therapeutically effective amount of ripretinib or a pharmaceutically acceptable salt thereof.

### CROSS-REFERENCE

This application is a continuation of U.S. Ser. No. 17/938,353 filed Oct. 6, 2022, which claims priority to U.S. Provisional Application No. 63/403,444 filed Sep. 2, 2022, each of which is incorporated herein by reference in its entirety.

### BACKGROUND

Gastrointestinal stromal tumors (GIST) comprise less than 1% of all gastrointestinal (GI) tumors, but constitute the most common mesenchymal tumors and soft tissue sarcomas of the GI tract. They occur anywhere along the GI tract but are found most often in the stomach (60%) or small intestine (30%) and less frequently in the rectum, colon, or mesentery. In the United States, around 3300 to 6000 new cases of GIST are diagnosed each year. The vast majority of cases are sporadic, and older age is a recognized risk factor. Mutations in KIT and platelet-derived growth factor receptor-alpha (PDGFRA) are found in over 80% of all primary GISTs. Alterations in neurofibromatosis type 1 gene (NF1) and succinate dehydrogenase (SDH) complex (SDHC) genes as well as altered methylation of SDHC promoter have been described as oncogenic drivers in GIST without activating mutations in KIT or PDGFRA, and they have been linked to familial and heritable syndromes (NF1 and Carney-Stratakis syndrome).

Despite a wide variation in tumor size, location, and histologic subtypes (spindle cell, epithelioid cells, and mixed type), approximately 85% of all GISTs share oncogenic mutations in 1 of 2 receptor tyrosine kinases (TKs): KIT or PDGFRA. Constitutive activation of either of these TKs plays a central role in the oncogenic behavior of GIST. The early characterization of GIST mutational status is important in both the localized and metastatic settings to identify imatinib-resistant mutations (such as some primary KIT exon 17 mutations or PDGFRA D842V) or mutations that require a higher dose of imatinib. Patients with GIST lacking KIT or PDGFRA mutations usually do not benefit from imatinib, and standard treatment algorithms mostly do not apply. However, other mutations may be present in these patients, with the largest group represented by SDH-deficiency frequently associated with Carney or Carney-Stratakis-Syndrome. Other subtypes have mutations in NF1 (usually associated with neurofibromatosis type I) or in BRAF or KRAS. Very recently, casuistic cases of GIST-like tumors harboring NTRK translocations have further expanded the spectrum of molecular subtypes.

In the pre-tyrosine kinase inhibitor (TKI) era, GISTs (often categorized as gastric leiomyosarcomas or leiomyoblastomas) were treated within the subtype of agnostic sarcoma trials and lacked an effective systemic therapy. However, a deeper understanding of the molecular pathogenesis and driving role of the protooncogenes KIT and PDGFRA has transformed the treatment of both localized and metastatic diseases. Localized and resectable tumors are treated surgically which remains the mainstay of curative therapy for localized disease. Resected high-risk GIST is typically treated with adjuvant imatinib, whereas low-risk GIST is managed with surgery alone. Intermediate-risk GIST is managed on a per-case basis. In an advanced/metastatic setting, imatinib 400 mg daily is approved, with dose escalation to 800 mg at the time of progression, and has been shown to yield dramatic results in disease control. Imatinib-refractory patients are treated with sunitinib as a second-line therapy and regorafenib as third-line therapy on resistance or intolerance to sunitinib.

At diagnosis, a mutation in the KIT gene occurs in 80% of GISTs and is usually found in exon 11, and less commonly in exon 9. Both mechanisms cause ligand-independent receptor activation, which leads to uncontrolled cell growth and transformation. Primary mutations affect a loss-of-function mutation in the JM domain and lead to a shift in equilibrium toward a Type I active or on-state conformation of KIT and away from a Type II inactive or off-state conformation of KIT. Exon 11 primary mutations are the most commonly seen in GISTs (around 70% of cases), and derive significant benefit from treatment with imatinib in both the adjuvant and metastatic settings, achieving a 2-year relapse-free survival of ˜90% in the adjuvant setting, and a median event-free survival just under 2 years in the metastatic setting. Primary mutations (in treatment-naïve patients) in exon 9 affect the extracellular domain of KIT, mimicking conformational changes induced by ligand binding and triggering KIT receptor homodimerization. This dimerization leads to the activation of specific intracellular signaling pathways which can lead to cancer cell proliferation, survival, and resistance. Although less common than exon 11 mutations, exon 9 mutations (10%-15% of newly diagnosed cases) are most commonly seen in GISTs arising from the small intestine. Unlike exon 11 mutations, they benefit less from imatinib in both the adjuvant and metastatic settings.

Despite significant improvement in outcomes compared with those in the pre-mutation-driven/TKI therapy era, response to imatinib is not experienced by all patients, and most patients with GIST will ultimately develop resistance to imatinib, most commonly due to the development of secondary mutations in KIT. Secondary resistance mutations usually arise in the catalytic domain of the kinase: 1) at the switch pocket, which typically occur in KIT exons 13 and 14 or PDGFRA exons 14 and 15 and sterically disrupt drug binding or conformationally activate KIT, and 2) in the activation loop switch encoded by KIT exons 17 and 18 and PDGFRA 18. Activation loop mutations act by shifting the kinase into an activated Type I or on-state conformation that is less amenable to drug binding by any of the approved Type II TKIs. Although uncommon in primary GIST (1%-2% of newly diagnosed cases), mutations in exons 13, 14 and 17 are often responsible for acquired imatinib resistance, with exon 17 mutations alone accounting for as many as 50% of the acquired resistance cases to imatinib, and later to sunitinib. A need exists for a TKI that can broadly inhibit clinically relevant KIT and PDGFRA mutations.

### SUMMARY

Disclosed herein, in various embodiments, is a method for treating a patient having a pre-existing Child-Pugh class C severe hepatic impairment and suffering from advanced gastrointestinal stromal tumor, comprising administering to the patient in need thereof a decreased dose of 50 mg of ripretinib once daily as compared to a dose of 150 mg once daily of ripretinib for patients with no pre-existing hepatic impairment.

Also disclosed herein, in various embodiments, is a method for treating a patient having a pre-existing Child-Pugh class C severe hepatic impairment and in need of treatment for advanced gastrointestinal stromal tumor; comprising orally administering to the patient 50 mg of ripretinib daily.

### BRIEF DESCRIPTION OF THE DRAWINGS

FIG. 1 depicts a plot of survival probability with respect to progression-free survival (PFS) for patients on ripretinib and patients on placebo as described in Example 1.

FIG. 2 depicts a plot of survival probability with respect to overall survival (OS) for patients on ripretinib and patients on placebo as described in Example 1.

FIG. 3 depicts plots illustrating survival probability with respect to OS in patients on ripretinib, crossed over from placebo to ripretinib, and patients without cross-over.

FIG. 4 depicts PFS by line of therapy for patients with GIST treated with 150 mg once daily of ripretinib.

FIG. 5A depicts patient report outcome by EQ-VAS visual scale, FIG. 5B depicts score changes from baseline used to assess these scores, and FIG. 5C depicts corresponding patient percentage distributions used in the study of Example 1 at 150 mg ripretinib QD. In the study, 70 patients were receiving ripretinib and 32 were receiving placebo.

FIG. 6A depicts EORTC QLQ-C30 physical function questions, FIG. 6B depicts patient score changes from baseline in response, and FIG. 6C depicts corresponding patient percentage distributions used in the study of Example 1 at 150 mg ripretinib QD. Physical function scores improved on average 1.6 from baseline to C2D1 among patients taking ripretinib in contrast to placebo patients who saw on average, a decline from baseline to C2D1 of 8.9 (p=0.004). In this study, 71 patients were receiving ripretinib and 32 were receiving placebo.

FIG. 7A depicts EORTC QLQ-C30 role function questions, FIG. 7B depicts patient score changes from baseline in response, and FIG. 7C depicts corresponding patient percentage distributions used in the study of Example 1 at 150 mg ripretinib QD. In the study, 70 patients were receiving ripretinib and 32 were receiving placebo.

FIG. 8A depicts patient score changes from baseline, and FIG. 8B depicts percentage distributions in response to Question C29 of EORTC QLQ-C30 (“How would you rate your overall health during the past week?”) from a scale of 1 (“Very poor”) to 7 (“Excellent”) in the study of Example 1 at 150 mg ripretinib QD. In the study, 70 patients were receiving ripretinib and 32 were receiving placebo.

FIG. 9A depicts patient score changes from baseline and FIG. 9B depicts corresponding patient percentage distributions in response to Question C30 of EORTC QLQ-C30 (“How would you rate your overall quality of life during the past week?”) from a scale of 1 (“Very poor”) to 7 (“Excellent”) in the study of Example 1 at 150 mg ripretinib QD. In the study, 70 patients were receiving ripretinib and 32 were receiving placebo.

FIG. 10 depicts mean changes in baseline scores in EQ-VAS across various time points, from Cycle 1, Day 15 up to Cycle 15, Day 1 of the intention-to-treat population in the study of Example 1 at 150 mg ripretinib QD.

FIG. 11A and FIG. 11B depict mean changes in baseline scores in EORTC QLQ-C30 role function and EORTC QLQ-C30 physical function, respectively, across various time points, from Cycle 1, Day 15 up to Cycle 15, Day 1 of the intention-to-treat population in the study of Example 1 at 150 mg ripretinib QD.

FIG. 12A and FIG. 12B depict mean changes in baseline scores in EORTC QLQ-C30 question C29 response and EORTC QLQ-C30 question C30 response, respectively, across various time points, from Cycle 1, Day 15 up to Cycle 15, Day 1 of the intention-to-treat population in the study of Example 1 at 150 mg ripretinib QD.

FIG. 13 depicts exemplary PFS data for patients who crossed over from placebo to ripretinib in the study described in Example 1 at 150 mg ripretinib QD.

FIG. 14A and FIG. 14B depict progression free survival (PFS) studies in the double-blind and open-label periods in the study of Example 1 for patients who dose escalated from 150 mg ripretinib QD 150 mg ripretinib BID. FIG. 14C depicting median PFS data and other parameters in these PFS studies.

FIG. 15 shows exemplary PFS data among patients with wild-type KIT mutations in the study of Example 1.

FIG. 16A and FIG. 16B depict Progression free survival (PFS) and overall survival (OS) data based on patients with a primary Exon 11 mutation or those with a non-Exon 11 mutation in the study of Example 1 at 150 mg ripretinib QD.

FIG. 17A and FIG. 17B depict progression free survival (PFS) and overall survival (OS) data based on patients with a primary Exon 11 mutation or a primary Exon 9 mutation in the study of Example 1 at 150 mg ripretinib QD.

FIG. 18A and FIG. 18B depict progression free survival (PFS) and overall survival (OS) data based on patients with a primary Exon 11 mutation, or a primary Exon 9 mutation, or another mutation, or wild type (KIT and PDGFRA) in the study of Example 1 at 150 mg ripretinib QD.

FIG. 19A and FIG. 19B depict progression free survival (PFS) studies for patients with certain primary mutations (Exon 9 or Exon 11) who dose escalated from 150 mg QD ripretinib to 150 mg BID ripretinib for the double-blind and open-label periods, respectively, in the study of Example 1.

FIG. 20 shows exemplary progression free survival data for patients with other KIT mutations and PGDFR mutations in the study of Example 1 at 150 mg ripretinib QD.

FIG. 21 depicts the median first appearance and worst grade of alopecia and PPES in corresponding patients receiving ripretinib in the study described in Example 1.

FIG. 22A depicts a mean change from baseline for physical function patient reported outcome of the EORTC-QLQ-C30 in patients receiving ripretinib, with and without alopecia, in the study described in Example 1. FIG. 22B depicts a mean change from baseline for role function patient reported outcome of the EORTC-QLQ-C30 in patients receiving ripretinib, with and without alopecia, in the study described in Example 1. FIG. 22C depicts a mean change from baseline for physical function patient reported outcome of the EORTC-QLQ-C30 in patients receiving riprotinib, with and without palmar-plantar erythrodysesthesia syndrome (PPES), in the study described in Example 1. FIG. 22D depicts a mean change from baseline for role function patient reported outcome of the EORTC-QLQ-C30 in patients receiving ripretinib, with and without palmar-plantar erythrodysesthesia syndrome (PPES), in the study described in Example 1.

FIG. 23A depicts a mean change from baseline for overall health patient reported outcome of the EORTC-QLQ-C30 in patients receiving ripretinib, with and without alopecia, in the study described in Example 1. FIG. 23B depicts a mean change from baseline for overall quality of life patient reported outcome of the EORTC-QLQ-C30 in patients receiving riprctinib, with and without alopecia, in the study described in Example 1. FIG. 23C depicts a mean change from baseline for overall health patient reported outcome of the EORTC-QLQ-C30 in patients receiving ripretinib, with and without palmar-plantar erythrodysesthesia syndrome (PPES), in the study described in Example 1. FIG. 23D depicts a mean change from baseline for overall quality of life patient reported outcome of the EORTC-QLQ-C30 in patients receiving ripretinib, with and without palmar-plantar erythrodysesthesia syndrome (PPES), in the study described in Example 1.

FIG. 24A depicts a mean change from baseline for state of health (VAS) patient reported outcome of the EORTC-QLQ-C30 in patients receiving ripretinib, with and without alopecia, in the study described in Example 1. FIG. 24B depicts a mean change from baseline for state of health (VAS) patient reported outcome of the EORTC-QLQ-C30 in patients receiving ripretinib, with and without palmar-plantar erythrodysesthesia syndrome (PPES), in the study described in Example 1.

FIG. 25A and FIG. 25B depict exemplary PFS of subjects who dose escalated to 150 mg BID, PFS before (PFS1) and after (PFS2) dose escalation.

FIG. 26A depicts an exemplary comparison of PFS of patient subgroups with Exon 9 KIT mutations. FIG. 26B depicts an exemplary comparison of PFS of patient subgroups with Exon 11 KIT mutations. FIG. 26C depicts an exemplary comparison of PFS of patient subgroups with Exon 13 KIT mutations. FIG. 26D depicts an exemplary comparison of PFS of patient subgroups with Exon 17 KIT mutations. This exemplary data show that ripretinib showed PFS benefit in all assessed patient subgroups compared to placebo.

FIG. 27 shows PFS data of exemplary second and third-line patients with Exons 9, 11, 13, or 17 KIT mutations. The data show that, in second and third line populations, patients with such various KIT mutations have comparable PFS.

FIG. 28 depicts a workflow of PBPK model development, verification, and application for ripretinib in the study of Example 11.

FIG. 29 depicts simulated and observed individual plasma concentration-time profiles of ripretinib and DP-5439 following single oral doses of ripretinib of 50 or 150 mg in the study of Example 11.

FIG. 30 depicts a comparison of the predicted and observed PK parameters for all model development and verification results for ripretinib and DP-5439 in the study of Example 11.

FIG. 31 depicts simulated mean plasma concentration-time profiles of ripretinib after multiple oral doses in the fasted state in healthy (150 mg QD) and severe HI (50 mg QD) groups in the study of Example 11.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

As used herein, “ripretinib” is a compound represented by the following structure:

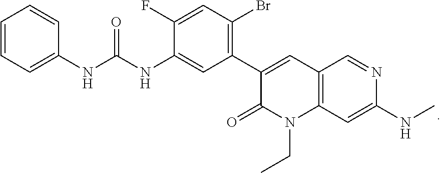 [SMILES: CCn1c(=O)c(-c2cc(NC(=O)Nc3ccccc3)c(F)cc2Br)cc2cnc(NC)cc21]

As used herein, “sunitinib” is a compound represented by the following structure:

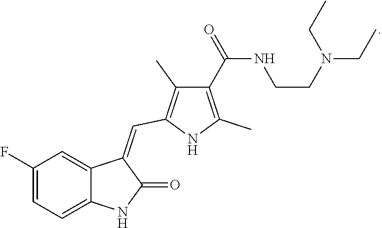 [SMILES: CCN(CC)CCNC(=O)c1c(C)[nH]c(/C=C2\C(=O)Nc3ccc(F)cc32)c1C]

As used herein, “imatinib” is a compound represented by the following structure:

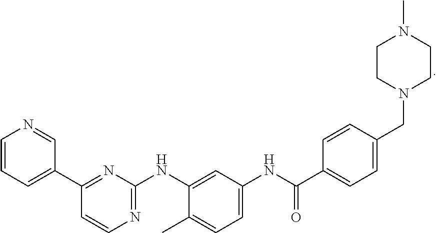 [SMILES: Cc1ccc(NC(=O)c2ccc(CN3CCN(C)CC3)cc2)cc1Nc1nccc(-c2cccnc2)n1]

As used herein, “regorafenib” is a compound represented by the following structure:

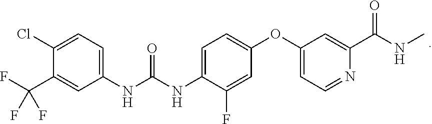 [SMILES: CNC(=O)c1cc(Oc2ccc(NC(=O)Nc3ccc(Cl)c(C(F)(F)F)c3)c(F)c2)ccn1]

As used herein, “Compound A” is a compound represented by the following structure:

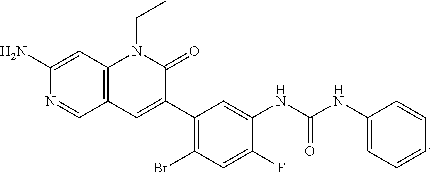 [SMILES: CCn1c(=O)c(-c2cc(NC(=O)Nc3ccccc3)c(F)cc2Br)cc2cnc(N)cc21]

Compound A is also referred to herein as DP-5439.

“Individual,” “patient,” or “subject” are used interchangeably herein and include any animal, including mammals, including mice, rats, other rodents, rabbits, dogs, cats, swine, cattle, sheep, horses, or primates, and humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods described herein is desirably a mammal in which treatment of a disorder described herein is desired, such as a human.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

As used herein, “treating” includes any effect, e.g., lessening, reducing, modulating, or eliminating, that results in the improvement of the condition, disease, disorder and the like.

“Therapeutically effective amount” includes the amount of the subject compound that will elicit the biological or medical response of a tissue, system, animal or human that is being sought by the researcher, veterinarian, medical doctor or other clinician. A compound described herein, e.g., ripretinib is administered in therapeutically effective amounts to treat a condition described herein, e.g., gastrointestinal stromal tumors. Alternatively, a therapeutically effective amount of a compound is the quantity required to achieve a desired therapeutic and/or prophylactic effect, such as an amount which results in the prevention of or a decrease in the symptoms associated with the condition.

As used herein, “AUC_{0-24 h}” refers to the area under the plasma concentration-time curve from time zero to 24 hours for a compound described herein. As used herein, “AUC_{0-inf}” refers to the area under the plasma concentration-time curve from time zero to infinite time for a compound described herein. As used herein, “Cₘₐₓ” refers to the maximum plasma concentration of a compound described herein.

A compound described herein, e.g., ripretinib, can be formulated as a pharmaceutical composition using a pharmaceutically acceptable carrier and administered by a variety of routes. In some embodiments, such compositions are for oral administration. In some embodiments, compositions formulated for oral administration are provided as tablets. In some embodiments, such compositions are for parenteral (by injection) administration. In some embodiments, such compositions are for transdermal administration. In some embodiments, such compositions are for topical administration. In some embodiments, such compositions are for intravenous (IV) administration. In some embodiments, such compositions are for intramuscular (IM) administration. Such pharmaceutical compositions and processes for preparing them are well known in the art. See, e.g., REMINGTON: THE SCIENCE AND PRACTICE OF PHARMACY (A. Gennaro, et al., eds., 19^{th} ed., Mack Publishing Co., 1995).

### Methods of Treatment

Described herein are methods of treating gastrointestinal stromal tumors in a patient in need thereof. In one embodiment, provided herein is a method for treating a patient having a pre-existing Child-Pugh class C severe hepatic impairment and suffering from advanced gastrointestinal stromal tumor, comprising administering to the patient in need thereof a decreased dose of 50 mg of ripretinib once daily as compared to a dose of 150 mg once daily of ripretinib for patients with no pre-existing hepatic impairment. In some embodiments, the patient having the pre-existing severe hepatic impairment has a ripretinib AUC₀₋ₜ of about 160% higher compared to a ripretinib AUC₀₋ₜ of a patient having no pre-existing hepatic impairment. In some embodiments, the patient having the pre-existing severe hepatic impairment has a ripretinib AUC₀₋ₜ of about 163% higher compared to a ripretinib AUC₀₋ₜ of a patient having no pre-existing hepatic impairment. In some embodiments, the patient having the pre-existing severe hepatic impairment has a ripretinib Cₘₐₓ of about 24% lower compared to a ripretinib Cₘₐₓ of a patient having no pre-existing hepatic impairment.

In another embodiment, disclosed herein is a method for treating a patient having a pre-existing Child-Pugh class C severe hepatic impairment and in need of treatment for advanced gastrointestinal stromal tumor; comprising orally administering to the patient 50 mg of ripretinib daily.

In another embodiment, the present disclosure relates to a method of treating a patient suffering from an advanced gastrointestinal stromal tumor, comprising orally administering to the patient 100 mg or more of ripretinib daily, e.g., 100 mg to 5000 mg, e.g., 100 mg to 500 mg, 100 mg to 250 mg, e.g., 150 mg, wherein the patient's tumor has progressed from, or the patient was intolerant to, a previous first line administration of imatinib. In some embodiments, the method comprises administering to the patient 110 mg of ripretinib daily. In some embodiments, the method comprises administering to the patient 120 mg of ripretinib daily. In some embodiments, the method comprises administering to the patient 130 mg of ripretinib daily. In some embodiments, the method comprises administering to the patient 140 mg of riprctinib daily. In some embodiments, the method comprises administering to the patient 150 mg of ripretinib daily. In some embodiments, the method comprises administering to the patient 200 mg of ripretinib daily. In some embodiments, the method comprises administering to the patient 250 mg of ripretinib daily. In some embodiments, the method comprises administering to the patient 300 mg of ripretinib daily. In some embodiments, the method comprises administering to the patient 350 mg of ripretinib daily. In some embodiments, the method comprises administering to the patient 400 mg of ripretinib daily. In some embodiments, the method comprises administering to the patient 450 mg of ripretinib daily. In some embodiments, the method comprises administering to the patient 500 mg of ripretinib daily. In some embodiments, the method comprises administering to the patient 550 mg of ripretinib daily. In some embodiments, the method comprises administering to the patient 600 mg of ripretinib daily. In some embodiments, the method comprises administering to the patient 650 mg of ripretinib daily. In some embodiments, the method comprises administering to the patient 700 mg of ripretinib daily. In some embodiments, the method comprises administering to the patient 750 mg of ripretinib daily. In some embodiments, the method comprises administering to the patient 800 mg of ripretinib daily. In some embodiments, the method comprises administering to the patient 850 mg of ripretinib daily. In some embodiments, the method comprises administering to the patient 900 mg of ripretinib daily. In some embodiments, the method comprises administering to the patient 950 mg of ripretinib daily. In some embodiments, the method comprises administering to the patient 1000 mg of ripretinib daily. In some embodiments, the method comprises administering to the patient 150 mg of ripretinib once daily. In some embodiments, the method comprises administering to the patient 200 mg of ripretinib once daily. In some embodiments, the method comprises administering to the patient 250 mg of riprctinib once daily. In some embodiments, the method comprises administering to the patient 300 mg of ripretinib once daily. In some embodiments, the method comprises administering to the patient 350 mg of ripretinib once daily. In some embodiments, the method comprises administering to the patient 400 mg of ripretinib once daily. In some embodiments, the method comprises administering to the patient 450 mg of ripretinib daily. In some embodiments, the method comprises administering to the patient 500 mg of ripretinib once daily. In some embodiments, the method comprises administering to the patient 550 mg of ripretinib once daily. In some embodiments, the method comprises administering to the patient 600 mg of ripretinib once daily. In some embodiments, the method comprises administering to the patient 650 mg of ripretinib once daily. In some embodiments, the method comprises administering to the patient 700 mg of ripretinib once daily. In some embodiments, the method comprises administering to the patient 750 mg of ripretinib once daily. In some embodiments, the method comprises administering to the patient 800 mg of ripretinib once daily. In some embodiments, the method comprises administering to the patient 850 mg of ripretinib once daily. In some embodiments, the method comprises administering to the patient 900 mg of ripretinib once daily. In some embodiments, the method comprises administering to the patient 950 mg of ripretinib once daily. In some embodiments, the method comprises administering to the patient 1000 mg of ripretinib once daily. In some embodiments, the method comprises administering to the patient 150 mg of ripretinib twice daily. In some embodiments, the method comprises administering to the patient 200 mg of ripretinib twice daily. In some embodiments, the method comprises administering to the patient 250 mg of ripretinib twice daily. In some embodiments, the method comprises administering to the patient 300 mg of ripretinib twice daily. In some embodiments, the method comprises administering to the patient 350 mg of ripretinib twice daily. In some embodiments, the method comprises administering to the patient 400 mg of ripretinib twice daily. In some embodiments, the method comprises administering to the patient 450 mg of ripretinib twice daily. In some embodiments, the method comprises administering to the patient 500 mg of ripretinib twice daily.

In some embodiments, a patient was only previously treated with a first line administration of imatinib, e.g., only imatinib and no other therapeutic compounds had been administered to the patient before administration of ripretinib to the patient. For example, a patient was not previously administered with sunitinib and/or regorafenib, e.g., the patient was not previously given a second line administration of sunitinib therapy and/or a third-line administration of regorafenib therapy.

In some embodiments, the patient has a non-nodal tumor lesion of greater than or equal to 1.0 cm in the long axis or greater than or equal to double the slide thickness in the long axis, within 21 days prior to the first dose of ripretinib. Contemplated methods of treatment include administering ripretinib on a 42-day cycle, comprising daily administrations of ripretinib without administering sunitinib. After at least one 42-day cycle comprising daily administrations of ripretinib, the patient may have progression-free survival as measured using mRECIST v1.1. In some embodiments, the patient treated with daily administrations of ripretinib may have significant progression free survival (e.g., about 3 months progression free survival or more, e.g., about 6 months progression free survival, as compared to a second line daily administration of 50 mg sunitinib for four weeks followed by two weeks without daily administrations on a 42-day cycle, wherein the patient's tumor has progressed from, or the patient was intolerant to, the previous first line administration of imatinib.

Contemplated treatments with ripretinib may treat a broad spectrum of KIT and PDGFRA mutations. For example, a patient's tumor may have a KIT exon 9 mutation, a PDGFRA exon 18 mutation, a PDGFRA exon 12 mutation or a PDGFRA exon 18 activation loop mutation. For example, the patient's tumor mutation is a PDGFRA D842V mutation.

In some embodiments, a patient's tumor has an imatinib resistant mutation selected from the group consisting of a KIT exon 17 activation loop mutation, a KIT exon 18 activation loop mutation, a KIT exon 13 mutation, a KIT exon 14 mutation, a KIT exon 18 mutation, a PDGFRA exon 12 mutation, a PDGFRA exon 14 mutation, a PDGRFA exon 15 mutation, and a PDGFRA exon 18 activation loop mutation. For example, the imatinib resistant mutation is a PDGFRA D842V mutation.

In some embodiments, the patient's tumor has an imatinib resistant mutation selected from the group consisting of KIT exon 13 or 14 mutation, PDGFRA exon 14 or 15 mutation, a KIT 17 or 18 activation loop mutation, and a PDGFRA 18 activation loop mutation. For example, the patient's tumor has an imatinib resistant KIT exon 17 mutation.

Also described herein is a method of treating a patient suffering from an advanced gastrointestinal stromal tumor, comprising orally administering to the patient 100 mg or more, e.g., up to about 600 mg, e.g. up to about 250 mg, e.g., 100 mg or 150 mg, of ripretinib daily, wherein the patient's tumor has progressed from, or the patient was intolerant to, a first line administration of imatinib, a second line administration of sunitinib, and a third line administration of regorafenib or wherein the patient has a documented intolerance to one or more of imatinib, sunitinib and/or regorafenib. Contemplated methods of treatment include orally administering 100 mg, 150 mg or more of ripretinib daily without administering sunitinib on a 42-day cycle. In some embodiments, the method comprises administering to the patient 110 mg of ripretinib daily. In some embodiments, the method comprises administering to the patient 120 mg of ripretinib daily. In some embodiments, the method comprises administering to the patient 130 mg of ripretinib daily. In some embodiments, the method comprises administering to the patient 140 mg of ripretinib daily. In some embodiments, the method comprises administering to the patient 150 mg of ripretinib daily. In some embodiments, the method comprises administering to the patient 200 mg of ripretinib daily. In some embodiments, the method comprises administering to the patient 250 mg of ripretinib daily. In some embodiments, the method comprises administering to the patient 300 mg of ripretinib daily. In some embodiments, the method comprises administering to the patient 350 mg of ripretinib daily. In some embodiments, the method comprises administering to the patient 400 mg of ripretinib daily. In some embodiments, the method comprises administering to the patient 450 mg of ripretinib daily. In some embodiments, the method comprises administering to the patient 500 mg of ripretinib daily. In some embodiments, the method comprises administering to the patient 550 mg of ripretinib daily. In some embodiments, the method comprises administering to the patient 600 mg of ripretinib daily. In some embodiments, the method comprises administering to the patient 650 mg of ripretinib daily. In some embodiments, the method comprises administering to the patient 700 mg of ripretinib daily. In some embodiments, the method comprises administering to the patient 750 mg of ripretinib daily. In some embodiments, the method comprises administering to the patient 800 mg of ripretinib daily. In some embodiments, the method comprises administering to the patient 850 mg of ripretinib daily. In some embodiments, the method comprises administering to the patient 900 mg of ripretinib daily. In some embodiments, the method comprises administering to the patient 950 mg of ripretinib daily. In some embodiments, the method comprises administering to the patient 1000 mg of ripretinib daily. In some embodiments, the method comprises administering to the patient 150 mg of ripretinib once daily. In some embodiments, the method comprises administering to the patient 200 mg of ripretinib once daily. In some embodiments, the method comprises administering to the patient 250 mg of ripretinib once daily. In some embodiments, the method comprises administering to the patient 300 mg of ripretinib once daily. In some embodiments, the method comprises administering to the patient 350 mg of ripretinib once daily. In some embodiments, the method comprises administering to the patient 400 mg of ripretinib once daily. In some embodiments, the method comprises administering to the patient 450 mg of ripretinib daily. In some embodiments, the method comprises administering to the patient 500 mg of ripretinib once daily. In some embodiments, the method comprises administering to the patient 550 mg of ripretinib once daily. In some embodiments, the method comprises administering to the patient 600 mg of ripretinib once daily. In some embodiments, the method comprises administering to the patient 650 mg of ripretinib once daily. In some embodiments, the method comprises administering to the patient 700 mg of ripretinib once daily. In some embodiments, the method comprises administering to the patient 750 mg of ripretinib once daily. In some embodiments, the method comprises administering to the patient 800 mg of ripretinib once daily. In some embodiments, the method comprises administering to the patient 850 mg of ripretinib once daily. In some embodiments, the method comprises administering to the patient 900 mg of ripretinib once daily. In some embodiments, the method comprises administering to the patient 950 mg of ripretinib once daily. In some embodiments, the method comprises administering to the patient 1000 mg of ripretinib once daily. In some embodiments, the method comprises administering to the patient 150 mg of ripretinib twice daily. In some embodiments, the method comprises administering to the patient 200 mg of ripretinib twice daily. In some embodiments, the method comprises administering to the patient 250 mg of ripretinib twice daily. In some embodiments, the method comprises administering to the patient 300 mg of ripretinib twice daily. In some embodiments, the method comprises administering to the patient 350 mg of ripretinib twice daily. In some embodiments, the method comprises administering to the patient 400 mg of ripretinib twice daily. In some embodiments, the method comprises administering to the patient 450 mg of ripretinib twice daily. In some embodiments, the method comprises administering to the patient 500 mg of ripretinib twice daily.

In some embodiments, a method of treating a patient suffering from an advanced gastrointestinal stromal tumor, comprising orally administering to the patient 100 mg or more of ripretinib daily, e.g., up to about 600 mg, e.g., 100 mg to 250 mg, e.g., 100 mg to 500 mg, e.g., 100 mg to 250 mg, e.g., 150 mg, wherein the patient was previously administered at least two tyrosine kinase inhibitors, is contemplated. Contemplated methods of treatment include orally administering 100 mg, 150 mg or more of ripretinib daily without administering sunitinib on a 42-day cycle. In some embodiments, the method comprises administering to the patient 110 mg of ripretinib daily. In some embodiments, the method comprises administering to the patient 120 mg of ripretinib daily. In some embodiments, the method comprises administering to the patient 130 mg of ripretinib daily. In some embodiments, the method comprises administering to the patient 140 mg of ripretinib daily. In some embodiments, the method comprises administering to the patient 150 mg of ripretinib daily. In some embodiments, the method comprises administering to the patient 200 mg of ripretinib daily. In some embodiments, the method comprises administering to the patient 250 mg of ripretinib daily. In some embodiments, the method comprises administering to the patient 300 mg of ripretinib daily. In some embodiments, the method comprises administering to the patient 350 mg of ripretinib daily. In some embodiments, the method comprises administering to the patient 400 mg of ripretinib daily. In some embodiments, the method comprises administering to the patient 450 mg of ripretinib daily. In some embodiments, the method comprises administering to the patient 500 mg of ripretinib daily. In some embodiments, the method comprises administering to the patient 550 mg of ripretinib daily. In some embodiments, the method comprises administering to the patient 600 mg of ripretinib daily. In some embodiments, the method comprises administering to the patient 650 mg of ripretinib daily. In some embodiments, the method comprises administering to the patient 700 mg of ripretinib daily. In some embodiments, the method comprises administering to the patient 750 mg of ripretinib daily. In some embodiments, the method comprises administering to the patient 800 mg of ripretinib daily. In some embodiments, the method comprises administering to the patient 850 mg of ripretinib daily. In some embodiments, the method comprises administering to the patient 900 mg of ripretinib daily. In some embodiments, the method comprises administering to the patient 950 mg of ripretinib daily. In some embodiments, the method comprises administering to the patient 1000 mg of ripretinib daily. In some embodiments, the method comprises administering to the patient 100 mg of ripretinib once daily. In some embodiments, the method comprises administering to the patient 150 mg of ripretinib once daily. In some embodiments, the method comprises administering to the patient 200 mg of ripretinib once daily. In some embodiments, the method comprises administering to the patient 250 mg of ripretinib once daily. In some embodiments, the method comprises administering to the patient 300 mg of ripretinib once daily. In some embodiments, the method comprises administering to the patient 350 mg of ripretinib once daily. In some embodiments, the method comprises administering to the patient 400 mg of ripretinib once daily. In some embodiments, the method comprises administering to the patient 450 mg of ripretinib daily. In some embodiments, the method comprises administering to the patient 500 mg of ripretinib once daily. In some embodiments, the method comprises administering to the patient 550 mg of ripretinib once daily. In some embodiments, the method comprises administering to the patient 600 mg of ripretinib once daily. In some embodiments, the method comprises administering to the patient 650 mg of ripretinib once daily. In some embodiments, the method comprises administering to the patient 700 mg of ripretinib once daily. In some embodiments, the method comprises administering to the patient 750 mg of ripretinib once daily. In some embodiments, the method comprises administering to the patient 800 mg of ripretinib once daily. In some embodiments, the method comprises administering to the patient 850 mg of ripretinib once daily. In some embodiments, the method comprises administering to the patient 900 mg of ripretinib once daily. In some embodiments, the method comprises administering to the patient 950 mg of ripretinib once daily. In some embodiments, the method comprises administering to the patient 1000 mg of ripretinib once daily. In some embodiments, the method comprises administering to the patient 150 mg of ripretinib twice daily. In some embodiments, the method comprises administering to the patient 200 mg of ripretinib twice daily. In some embodiments, the method comprises administering to the patient 250 mg of ripretinib twice daily. In some embodiments, the method comprises administering to the patient 300 mg of ripretinib twice daily. In some embodiments, the method comprises administering to the patient 350 mg of ripretinib twice daily. In some embodiments, the method comprises administering to the patient 400 mg of ripretinib twice daily. In some embodiments, the method comprises administering to the patient 450 mg of ripretinib twice daily. In some embodiments, the method comprises administering to the patient 500 mg of ripretinib twice daily. In some embodiments, the patient has previously been administered two separate tyrosine kinase inhibitors, each selected from the group consisting of imatinib, sunitinib, regorafenib, lapatinib, gefitinib, erlotinib, vatalanib, crenolanib, and pharmaceutically acceptable salts thereof. In some embodiments, each of the tyrosine kinase inhibitors is independently selected from the group consisting of imatinib, sunitinib, and regorafenib. In some embodiments, each of the tyrosine kinase inhibitors is independently selected from the group consisting of imatinib mesylate, sunitinib malate, and regorafenib.

In some embodiments of the methods described herein, the patient is orally administered one or more tablets comprising ripretinib. For example, the disclosed methods include a method of treating a patient suffering from an advanced gastrointestinal stromal tumor, comprising orally administering to the patient one or more tablets comprising ripretinib, e.g., tablets each comprising 50 mg to 100 mg of ripretinib, daily, wherein the patient's tumor has progressed from, or the patient was intolerant to, a previous first line administration of imatinib. In some embodiments, the patient is administered one tablet comprising ripretinib. In some embodiments, the patient is administered one tablet comprising 50 mg of ripretinib. In some embodiments, the patient is administered one tablet comprising 50 mg of ripretinib once daily.

In some embodiments, the patient is administered two tablets each comprising 50 mg of ripretinib. In some embodiments, the patient is administered two tablets each comprising 50 mg of ripretinib once daily. In some embodiments, the patient is administered three tablets each comprising 50 mg of ripretinib. In some embodiments, the patient is administered three tablets each comprising 50 mg of ripretinib once daily. In some embodiments, the patient is administered four tablets each comprising 50 mg of ripretinib. In some embodiments, the patient is administered four tablets each comprising 50 mg of ripretinib once daily. In some embodiments, the patient is administered five tablets each comprising 50 mg of ripretinib. In some embodiments, the patient is administered five tablets each comprising 50 mg of ripretinib once daily. In some embodiments, the patient is administered six tablets each comprising 50 mg of ripretinib. In some embodiments, the patient is administered six tablets each comprising 50 mg of ripretinib once daily.

Additionally, the disclosed methods include a method of treating a patient suffering from an advanced gastrointestinal stromal tumor, comprising orally administering to the patient, on a daily basis, one or more tablets each comprising ripretinib, e.g., tablets each comprising 50 mg to 100 mg of ripretinib, wherein the patient's tumor has progressed from, or the patient was intolerant to, a first line administration of imatinib, a second line administration of sunitinib, and a third line administration of regorafenib or wherein the patient has a documented intolerance to one or more of imatinib, sunitinib and/or regorafenib. In some embodiments, the patient is administered one tablet comprising ripretinib. In some embodiments, the patient is administered one tablet comprising 50 mg of ripretinib. In some embodiments, the patient is administered one tablet comprising 50 mg of ripretinib once daily. In some embodiments, the patient is administered two tablets each comprising 50 mg of ripretinib. In some embodiments, the patient is administered, once daily, two tablets each comprising 50 mg of ripretinib. In some embodiments, the patient is administered three tablets each comprising 50 mg of ripretinib. In some embodiments, the patient is administered, once daily, three tablets each comprising 50 mg of ripretinib once daily.

In some embodiments, provided is a method of treating a patient suffering from an advanced gastrointestinal stromal tumor, comprising orally administering to the patient, on a daily basis, one or more tablets each comprising ripretinib, e.g., tablets each comprising 50 mg to 100 mg of ripretinib, wherein the patient was previously administered at least two tyrosine kinase inhibitors before administration of the ripretinib. In some embodiments, the patient is administered one tablet comprising ripretinib. In some embodiments, the patient is administered one tablet comprising 50 mg of ripretinib. In some embodiments, the patient is administered one tablet comprising 50 mg of ripretinib once daily. In some embodiments, the patient is administered two tablets each comprising 50 mg of ripretinib. In some embodiments, the patient is administered, once daily, two tablets each comprising 50 mg of ripretinib. In some embodiments, the patient is administered three tablets each comprising 50 mg of ripretinib. In some embodiments, the patient is administered, once daily, three tablets each comprising 50 mg of ripretinib. In some embodiments, the patient has previously been administered two separate tyrosine kinase inhibitors, each selected from the group consisting of imatinib, sunitinib, regorafenib, lapatinib, gefitinib, erlotinib, vatalanib, crenolanib, and pharmaceutically acceptable salts thereof. In some embodiments, each of the tyrosine kinase inhibitors is independently selected from the group consisting of imatinib, sunitinib, and regorafenib. In some embodiments, each of the tyrosine kinase inhibitors is independently selected from the group consisting of imatinib mesylate, sunitinib malate, and regorafenib.

In another embodiment, described herein is a method of treating a patient suffering from an advanced gastrointestinal stromal tumor, comprising orally administering to the patient 150 mg of ripretinib once daily, wherein the patient was previously administered three or more kinase inhibitors before administration of the ripretinib. In some embodiments, after at least 4 weeks of the daily ripretinib administration, the patient has at least a 5-month progression-free survival as measured using mRECIST v1.1. In some embodiments, orally administering to the patient 150 mg of ripretinib once daily comprises administering to the patient three tablets each tablet comprising 50 mg of ripretinib. In some embodiments, one of the three or more kinase inhibitors is imatinib. In some embodiments, the patient was previously administered imatinib, sunitinib and regorafenib.

In some embodiments, if the patient suffers from a Grade 3 palmer-plantar erythrodysesthia syndrome upon administration of the ripretinib, the method further comprises a) withholding administration of ripretinib for at least 7 days or until the patient has less than or equal to Grade 1 palmer-plantar erythrodysesthia syndrome, then administering to the patient 100 mg daily (e.g., 100 mg once daily) ripretinib for at least 28 days.

In some embodiments, if the patient suffers from a Grade 2 palmer-plantar erythrodysesthia syndrome upon administration of the ripretinib, the method further comprises: a) withholding administration of ripretinib until the patient has less than or equal to Grade 1 palmer-plantar erythrodysesthia syndrome or baseline; b) if the patient recovers from the palmer-plantar erythorysesthia syndrome within 7 days of withholding administration, then administering to the patient 150 mg daily ripretinib or c) if the patient has not recovered, then administering to the patient 100 mg daily ripretinib for at least 28 days.

In another embodiment, described herein is a method of treating a patient suffering from an advanced gastrointestinal stromal tumor, comprising orally administering to the patient 150 mg daily ripretinib, wherein the patient's tumor has progressed from, or the patient was intolerant to, a first line administration of imatinib, a second line administration of sunitinib, and a third line administration of regorafenib.

In another embodiment, described herein is a method of treating a patient suffering from an advanced gastrointestinal stromal tumor, comprising orally administering to the patient 150 mg of ripretinib once or twice daily, wherein the patient's tumor has progressed from, or the patient was intolerant to, a previous first line administration of imatinib. In some embodiments, if the patient suffers from Grade 3 palmer-plantar erythrodysesthia syndrome upon administration of the ripretinib, the method further comprises a) withholding administration of ripretinib for at least 7 days or until the patient has less than or equal to Grade 1 palmer-plantar erythrodysesthia syndrome, then administering to the patient 100 mg daily (e.g., 100 mg once daily) ripretinib for at least 28 days. In some embodiments, if the patient suffers from Grade 3 palmer-plantar erythrodysesthia syndrome upon administration of the ripretinib, the method further comprises a) withholding administration of ripretinib for at least 7 days or until the patient has less than or equal to Grade 1 palmer-plantar erythrodysesthia syndrome, then administering to the patient 100 mg daily (e.g., 100 mg once daily) ripretinib for at least 28 days. In some embodiments, if the patient suffers from Grade 2 palmer-plantar erythrodysesthia syndrome upon administration of the ripretinib, the method further comprises: a) withholding administration of ripretinib until the patient has less than or equal to Grade 1 palmer-plantar erythrodysesthia syndrome or baseline; b) if the patient recovers from the palmer-plantar erythorysesthia syndrome within 7 days of withholding administration, then administering to the patient 150 mg daily ripretinib or c) if the patient has not recovered, then administering to the patient 100 mg daily ripretinib for at least 28 days. In some embodiments, if the patient suffers from a Grade 3 adverse disorder selected from arthralgia or myalgia upon administration of the ripretinib, the method further comprises: a) withholding administration of ripretinib until the patient has less than or equal to Grade 1 adverse disorder, then administering to the patient 100 mg daily (e.g., 100 mg once daily) ripretinib for at least 28 days. In some embodiments, if the patient suffers from Grade 3 hypertension upon administration of the ripretinib, the method further comprises withholding administration of ripretinib until the patient's blood pressure is controlled, and if the patient has less than or equal to Grade 1 blood pressure is, administering to the patient 150 mg daily ripretinib, or if the patient has more than Grade 1 blood pressure, administering 100 mg daily (e.g., 100 mg once daily) ripretinib.

In another embodiment, described herein is a method for achieving at least 5 months of progression free survival as determined by mRECIST 1.1 in a patient having an advanced gastrointestinal stromal tumor, comprising orally administering to the patient 50, 100, 150 200, or 300 mg of ripretinib daily or twice daily for at least 28 days. In some embodiments, the patient has been administered at least one previous kinase inhibitor. In some embodiments, the patient has been administered at least three previous kinase inhibitors. In some embodiments, the at least one previous kinase inhibitor is imatinib. In some embodiments, comprising orally administering to the patient 50, 100, 150 or 200 mg of ripretinib daily or twice daily for at least 4 months.

In another embodiment, described herein is a method for achieving at least 5 months of progression free survival as determined by mRECIST 1.1 in a patient having an advanced gastrointestinal stromal tumor, comprising orally administering to the patient 50, 100, 150, or 200 mg of ripretinib daily or twice daily for at least 28 days. In some embodiments, the patient has been administered at least one previous kinase inhibitor. In some embodiments, the patient has been administered at least three previous kinase inhibitors. In some embodiments, the at least one previous kinase inhibitor is imatinib. In some embodiments, comprising orally administering to the patient 50, 100, 150, or 200 mg of ripretinib daily or twice daily for at least 4 months.

In another embodiment, described herein is a method of treating a gastrointestinal stromal tumor in a patient in need thereof, wherein the patient is being treated concurrently with a proton pump inhibitor, the method comprising: orally administering to the patient 100 mg or 150 mg of ripretinib, or a pharmaceutically acceptable salt thereof, once or twice daily, and wherein administration of the ripretinib and proton pump inhibitor to the patient provides no clinically significant difference in the plasma exposure of ripretinib in the patient as compared to administration of ripretinib without concurrent treatment of the proton pump inhibitor. In some embodiments, the proton pump inhibitor is selected from the group consisting of pantoprazole, omeprazole, lansoprazole, rabeprazole, esomeprazole, and dexlansoprazole. In some embodiments, the proton pump inhibitor is pantoprazole. In some embodiments, the patient is being treated concurrently with 40 mg of the proton pump inhibitor once daily.

In another embodiment, described herein is a method of treating a gastrointestinal stromal tumor in a patient in need thereof, the method comprising orally administering to the patient 50 mg, 100 mg, or 150 mg of ripretinib, or a pharmaceutically acceptable salt thereof, once or twice daily, wherein the ripretinib is administered to the patient with food or without food. In some embodiments, the food comprises a high-fat meal (e.g., a high-fat meal described herein).

In some embodiments, the therapeutic efficacy of ripretinib is determined by the progression-free survival of the patient after independent radiologic review using Response Evaluation Criteria in Solid Tumors (RECIST). In some embodiments, the therapeutic efficacy of ripretinib is determined by the progression-free survival of the patient after independent radiologic review using modified Response Evaluation Criteria in Solid Tumors (mRECIST). In some embodiments, the therapeutic efficacy of ripretinib is determined by the Objective Response Rate (ORR), Time to Tumor Progression (TTP) or Overall Survival (OS) of the patient after independent radiologic review using mRECIST. In some embodiments, the therapeutic efficacy of ripretinib is determined by the progression-free survival of the patient based on investigator assessment. In some embodiments, the therapeutic efficacy of ripretinib is determined by the quality of life of the patient in accordance with European Organisation for Research and Treatment of Cancer Quality of Life Questionnaire for Cancer 30-item (EORTC-QLQ-C30) and the EuroQol 5-Dimension 5-Level (EQ-5D-5L) questionnaires. In some embodiments, the therapeutic efficacy of ripretinib is determined by the disease control rate of the patient. In some embodiments, the therapeutic efficacy of ripretinib is determined by the duration of response of the patient.

After at least one month, two months, e.g., 42 days or more of treatment with ripretinib, the patient may have a progression-free survival as measured using mRECIST v1.1. As another example, the patient may have a least a 5 or 6 month progression-free survival as compared to placebo after at least 4 weeks of daily administration of ripretinib, and/or for example, after 4 weeks of daily administration of ripretinib, significantly reduced the risk of disease progression or death by 85%.

In some embodiments, the patient has at least one measurable tumor lesion according to modified RECIST Version 1.1 within 21 days prior to the first dose of ripretinib. In some embodiments, the patient has a non-nodal tumor lesion of greater than or equal to 1.0 cm in the long axis or greater than or equal to double the slide thickness in the long axis, within 21 days prior to the first dose of ripretinib.

In some embodiments, the patient's tumor has a KIT exon 9 mutation, a PDGFRA exon 18 mutation, a PDGFRA exon 12 mutation or a PDGFRA exon 18 activation loop mutation. For example, the patient's tumor mutation is a PDGFRA D842V mutation.

In some embodiments, the patient's tumor has an imatinib resistant, sunitinib resistant, and/or regorafenib resistant mutation selected from the group consisting of a KIT exon 17 activation loop mutation, a KIT exon 18 activation loop mutation, a KIT exon 13 mutation, a KIT exon 14 mutation, a KIT exon 18 mutation, a PDGFRA exon 12 mutation, a PDGFRA exon 14 mutation, a PDGRFA exon 15 mutation, and a PDGFRA exon 18 activation loop mutation. For example, the resistant mutation is a PDGFRA D842V mutation.

In some embodiments, the patient's tumor has a drug resistant mutation selected from the group consisting of KIT exon 13 or 14 mutation, PDGFRA exon 14 or 15 mutation, a KIT 17 or 18 activation loop mutation, and a PDGFRA 18 activation loop mutation. For example, the tumor has a drug resistant KIT exon 17 mutation.

### Dose Modifications

Dose modifications may be made in the methods of administering ripretinib described herein as a result of adverse events experienced by the patient or due to pre-existing conditions of the patient.

In some embodiments, the dose modification is a dose interruption. In some embodiments, the dose modification is a permanent discontinuation in dosing. In some embodiments, the dose modification is a dose reduction. In some embodiments, the dose of ripretinib administered to the patient is reduced from 150 mg once daily, e.g., three tablets each comprising 50 mg of ripretinib, to 100 mg once daily, e.g., two tablets each comprising 50 mg of ripretinib. In some embodiments, the dose of ripretinib administered to the patient is reduced from 150 mg once daily, e.g., three tablets each comprising 50 mg of ripretinib, to 50 mg once daily, e.g., one tablet comprising 50 mg of ripretinib. In some embodiments, the adverse reaction is selected from the group consisting of a hand-foot skin reaction (e.g., palmar-plantar erthrodysesthesia syndrome), hypertension, arthralgia, and myalgia.

In some embodiments, the adverse event is graded in accordance with the National Cancer Institute Common Terminology Criteria for Adverse Events, version 4.03 (e.g., baseline, Grade 1, Grade 2, Grade 3, Grade 4, or Grade 5). In some embodiments, the dose modification is a dose interruption (e.g., a dose interruption of at least 7 days) as a result of a Grade 2 adverse event. In some embodiments, dosing resumes at the same dose level before the dose interruption if the adverse event is lowered to Grade 1 or baseline within a first time period (e.g., within 7 days). In some embodiments, dosing resumes at a reduced dose level before the dose interruption if the adverse event is lowered to Grade 1 or baseline after a first time period (e.g., after 7 days). In some embodiments, the reduced dose level is re-escalated to the dose level prior to the dose interruption if the adverse event is lowered to Grade 1 or baseline after a first time period but is maintained as a Grade 1 or baseline adverse event after a second time period (e.g., after 28 days). In some embodiments, the dose modification is a dose interruption (e.g., a dose interruption of at least 7 days up to a maximum of 28 days) as a result of a Grade 3 adverse event. In some embodiments, dosing is continued at a reduced level after the dose interruption. In some embodiments, the dose modification is a permanent discontinuation in dosing as a result of a Grade 4 adverse event (e.g., Grade 4 hypertension).

A patient can be administered an additional treatment in response to an adverse event or to prevent an adverse event from occurring. In some embodiments, a patient suffering from an adverse dermatologic reaction, e.g., a hand-foot skin reaction, e.g., palmar-plantar erthrodysesthesia syndrome, is administered a topical composition (e.g., an emollient) to treat the adverse dermatologic reaction. In some embodiments, the patient is administered the topical composition (e.g., an emollient) based on the severity of the adverse dermatologic reaction, e.g., a Grade 2, Grade 3 adverse dermatologic reaction, e.g., a Grade 1, Grade 2, or Grade 3 hand-foot skin reaction, e.g., a Grade 1, Grade 2 or Grade 3 palmar-plantar erthrodysesthesia syndrome. In some embodiments, the topical composition (e.g., an emollient) is administered to the patient during a dose interruption of ripretinib. In some embodiments, the topical composition (e.g., an emollient) is administered to the patient contemporaneously with a dose of ripretinib, e.g., a reduced dose of ripretinib.

A patient can also be administered an additional treatment prior to, or during administration of ripretinib in accordance with the methods described herein to prevent or ameliorate an adverse event. In some embodiments, the patient is administered a topical composition (e.g., an emollient) before and/or during ripretinib administration to prevent or ameliorate the onset of an adverse dermatologic reaction, e.g., a hand-foot skin reaction, e.g., palmar-plantar erthrodysesthesia syndrome.

In some embodiments, a patient suffering from advanced gastrointestinal stromal tumor has a pre-existing medical condition. In some embodiments, the pre-existing medical condition is a hepatic impairment. In some embodiments, the pre-existing medical condition is a Child-Pugh class C severe hepatic impairment. In some embodiments, a patient having a pre-existing Child-Pugh class C severe hepatic impairment and suffering from advanced gastrointestinal stromal tumor is treated with a reduced dose of ripretinib. In some embodiments, the reduced dose comprises 100 mg or 50 mg ripretinib administered once daily compared to a dose of 150 mg once daily of ripretinib for patients with no pre-existing hepatic impairment. In some embodiments, the reduced dose comprises or 50 mg ripretinib administered once daily compared to a dose of 150 mg once daily of ripretinib for patients with no pre-existing hepatic impairment.

In some embodiments, a patient having a pre-existing Child-Pugh class C severe hepatic impairment and suffering from advanced gastrointestinal stromal tumor has increased AUC_{0-24 h} of ripretinib compared to a patient having no pre-existing hepatic impairment. In some embodiments, the patient having severe hepatic impairment has a ripretinib AUC₀₋ₜ of about 160% higher compared to a ripretinib AUC₀₋ₜ of a patient having no pre-existing hepatic impairment. In some embodiments, the patient having severe hepatic impairment has a ripretinib AUC₀₋ₜ of about 163% higher compared to a ripretinib AUC₀₋ₜ of a patient having no pre-existing hepatic impairment.

In some embodiments, the patient having a pre-existing Child-Pugh class C severe hepatic impairment and suffering from advanced gastrointestinal stromal tumor has a decreased Cₘₐₓ of ripretinib compared to a patient having no pre-existing hepatic impairment. In some embodiments, the patient having severe hepatic impairment has a ripretinib Cₘₐₓ of about 24% lower compared to a ripretinib Cₘₐₓ of a patient having no pre-existing hepatic impairment.

### EXAMPLES

### Example 1. A Randomized, Double-Blind, Placebo-Controlled, International, Multicenter Study to Evaluate the Safety, Tolerability, and Efficacy of Ripretinib Compared to Placebo in Patients with Advanced GIST Whose Previous Therapies have Included Imatinib, Sunitinib, and Regorafenib

This study was a randomized (2:1), double-blind, placebo-controlled, international, multicenter study to evaluate the safety, tolerability, and efficacy of ripretinib compared to placebo in 129 patients with advanced GIST whose previous therapies have included at least imatinib, sunitinib, and regorafenib. Patients were randomized 2:1 to either 150 mg of ripretinib or placebo once daily. The primary efficacy endpoint is progression-free survival (PFS) as determined by independent radiologic review using modified Response Evaluation Criteria in Solid Tumors (RECIST). Secondary endpoints as determined by independent radiologic review using modified RECIST include Objective Response Rate (ORR), Time to Tumor Progression (TTP) and Overall Survival (OS).

Results. This study achieved its primary endpoint of improved PFS as determined by blinded independent central radiologic review using modified Response Evaluation Criteria in Solid Tumors (RECIST) version 1.1.

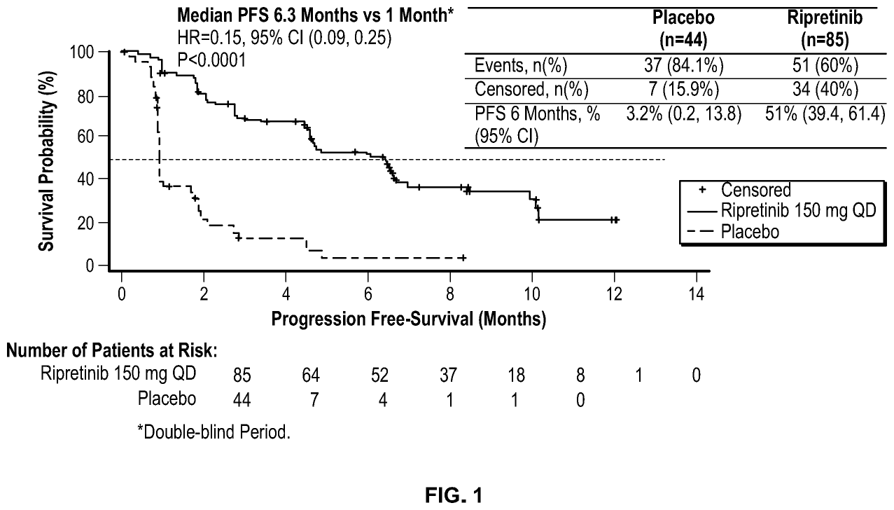

Ripretinib demonstrated a median PFS of 6.3 months (27.6 weeks) compared to 1.0 month (4.1 weeks) in the placebo arm and significantly reduced the risk of disease progression or death by 85% (HR of 0.15, p<0.0001) compared to placebo. PFS rates at 6 months were 51% (95% CI: 39.4, 61.4) for ripretinib and 3.2% (95% CI: 0.2, 13.8) for placebo. Plots of survival probability with respect to PFS for patients on ripretinib and patients on placebo are shown in FIG. 1.

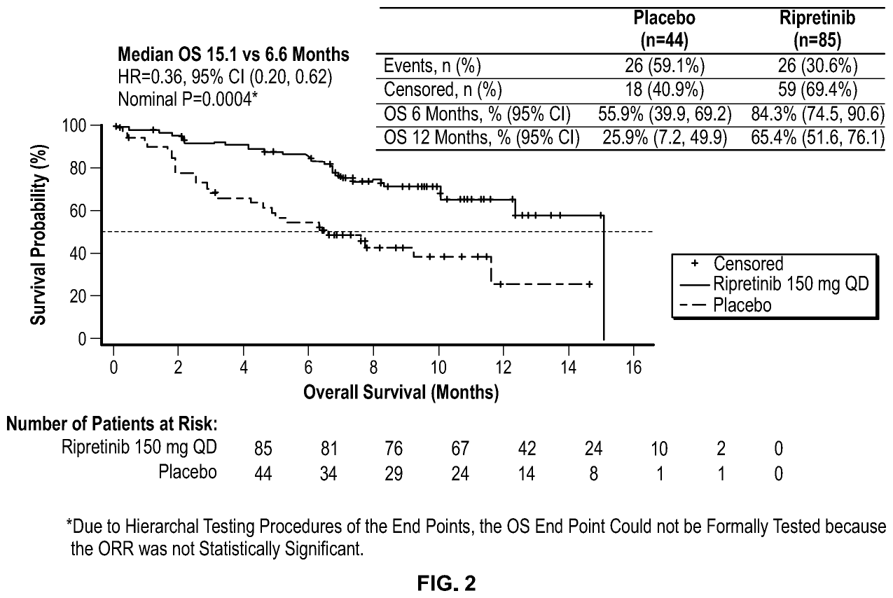

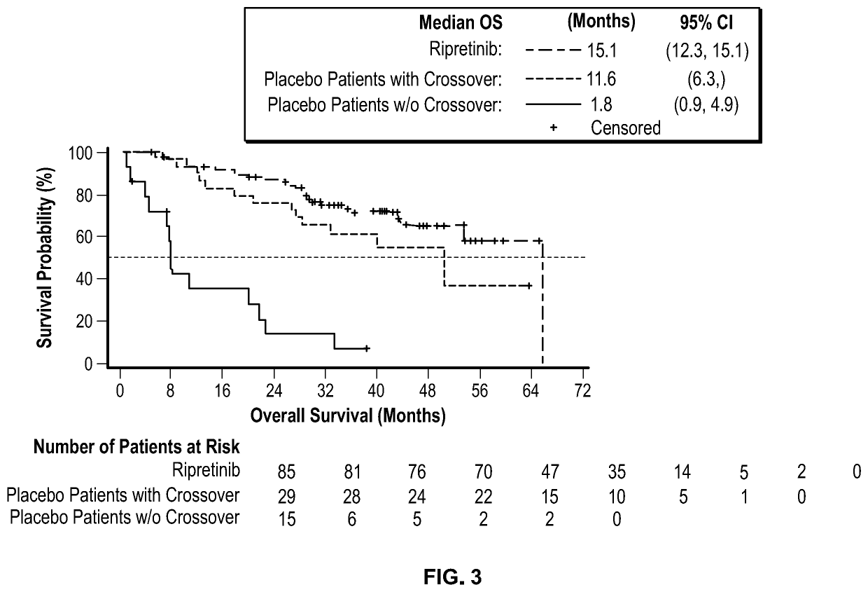

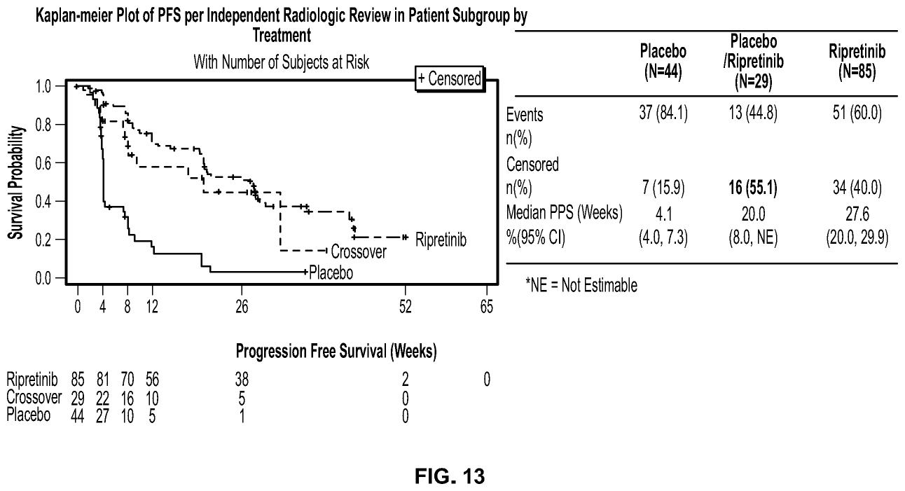

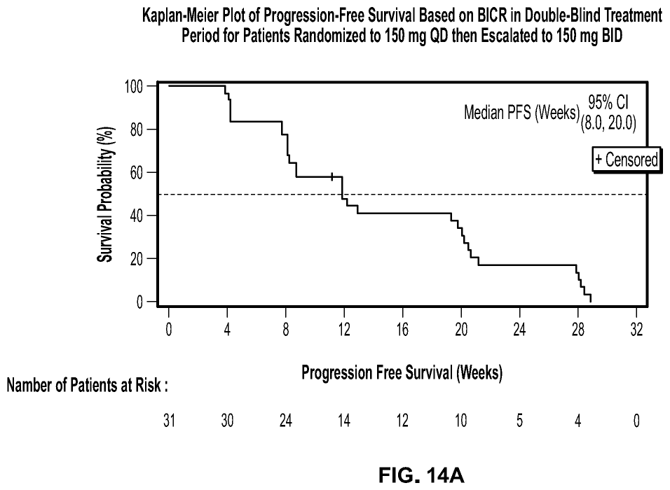

For the key secondary endpoint of objective response rate (ORR), as determined by blinded independent central radiologic review using modified RECIST version 1.1, ripretinib demonstrated an ORR of 9.4% compared with 0% for placebo (p-value=0.0504), which was not statistically significant. Ripretinib in this study also showed a clinically meaningful improvement over placebo in terms of the secondary endpoint overall survival (OS) (median OS 15.1 months vs. 6.6 months, HR=0.36, nominal p-value=0.0004; OS rates at 12 months were 65.4% (95% CI: 51.6, 76.1) for ripretinib and 25.9% (95% CI: 7.2, 49.9) for placebo); however, because statistical significance was not achieved for ORR, the hypothesis testing of OS was not formally performed. Plots of survival probability with respect to OS of patients on ripretinib and patients on placebo are shown in FIG. 2. According to the pre-specified hierarchical testing procedure of the endpoints, the hypothesis testing of OS cannot be formally conducted unless the test of ORR is statistically significant. The OS data for the placebo arm includes patients taking placebo who, following progression, were crossed-over to ripretinib treatment. Plots illustrating survival probability with respect to OS in patients on ripretinib, crossed over from placebo to ripretinib, and patients without cross-over are shown in FIG. 3. Plots and additional data illustrating survival probability with respect to PFS in patients on ripretinib, crossed over from placebo to ripretinib, and patients without cross-over are shown in FIG. 13. Additionally, more patients receiving ripretinib had stable disease (SD) for 12 weeks (40 [47·1%] vs 2 [4·5%], respectively) and less PD (16 [18·8%] vs 28 [63·6%], respectively) than patients on placebo. The large percentage of patients receiving ripretinib with stable disease (SD) is notable as the absence of progression is considered an important marker of therapeutic benefit in GIST. Unlike many other advanced solid tumors, the absence of progression (whether a partial response (PR) or SD) is predictive of PFS and OS benefit in patients with advanced GIST.

Patients that successfully crossed over from placebo had smaller tumors vs those that did not crossover (median sum of longest diameter of target lesions 119.4 mm vs 183.3 mm). In addition, the median age of those who crossed over was higher than those who did not cross over (68.0 vs 58.0 years) and none had a baseline Eastern Cooperative Oncology Group (ECOG) score of 2, while 3 patients in the group that did not cross over had an ECOG score of 2. Patients that crossed over had a median PFS of 20.0 weeks [95% CI, 8.0-NE] vs 27.6 weeks seen in the initial ripretinib arm and 4.1 weeks seen in the initial placebo arm, respectively. Median OS in patients who crossed over was 11.6 months, vs. 15.1 months in patients initially randomized to ripretinib and 1.8 months in patients on placebo who did not crossover. TEAEs during the crossover period suggest no safety concerns when compared to the safety profile of patients initially assigned to ripretinib.

Ripretinib was generally well tolerated and the adverse event results were consistent with data from previously presented Phase 1 study results. Grade 3 or 4 treatment-emergent adverse events (TEAEs) occurred in 42 (49%) patients on the ripretinib arm compared to 19 (44%) on the placebo arm. Grade 3 or 4 TEAEs>5% of patients in the ripretinib arm were anemia (9%; n=8), abdominal pain (7%; n=6) and hypertension (7%; n=6). Grade 3 or 4 TEAEs>5% of patients in the placebo arm were anemia (14%; n=6). Table 1 lists TEAEs>15% in the ripretinib arm compared to placebo.

TABLE 1
Treatment-emergent adverse events for patients
administered with either ripretinib or placebo.

| | | Ripretinib |
|---|---|---|
| | | 150 mg |
| Treatment Emergent | Placebo | Daily |
| Adverse Event | (N = 43)⁽¹⁾ | (N = 85)⁽¹⁾ |
| Any event | 42 (98%) | 84 (99%) |
| Alopecia | 2 (5%) | 44 (52%) |
| Fatigue | 10 (23%) | 36 (42%) |
| Nausea | 5 (12%) | 33 (39%) |
| Abdominal pain | 13 (30%) | 31 (36%) |
| Constipation | 8 (19%) | 29 (34%) |
| Myalgia | 5 (12%) | 27 (32%) |
| Diarrhea | 6 (14%) | 24 (28%) |
| Decreased appetite | 9 (21%) | 23 (27%) |
| Palmar-plantar | 0 | 18 (21%) |
| erythrodysaesthesia syndrome | | |
| Vomiting | 3 (7%) | 18 (21%) |
| Headache | 2 (5%) | 16 (19%) |
| Weight decreased | 5 (12%) | 16 (19%) |
| Arthralgia | 2 (5%) | 15 (18%) |
| Blood bilirubin increased | 0 | 14 (16%) |
| Oedema peripheral | 3 (7%) | 14 (16%) |
| Muscle spasms | 2 (5%) | 13 (15%) |

In table 1, (1) indicates that the safety population includes 128 patients. One patient was randomized to placebo but did not receive study drug.

### Patient Reported Outcomes

Patient reported outcomes were assessed by EQ-5D-5L, which provides a visual analogue scale (VAS), and EORTC QLQ-C30, which provides physical function and role function scales. The EQ-5D-5L VAS (or EQ-VAS) records the respondent's overall current health on a vertical visual analogue scale and provides a quantitative measure of the patient's perception of overall health. These patient outcomes were reported across 28-day cycles of receiving 150 mg QD ripretinib or placebo.

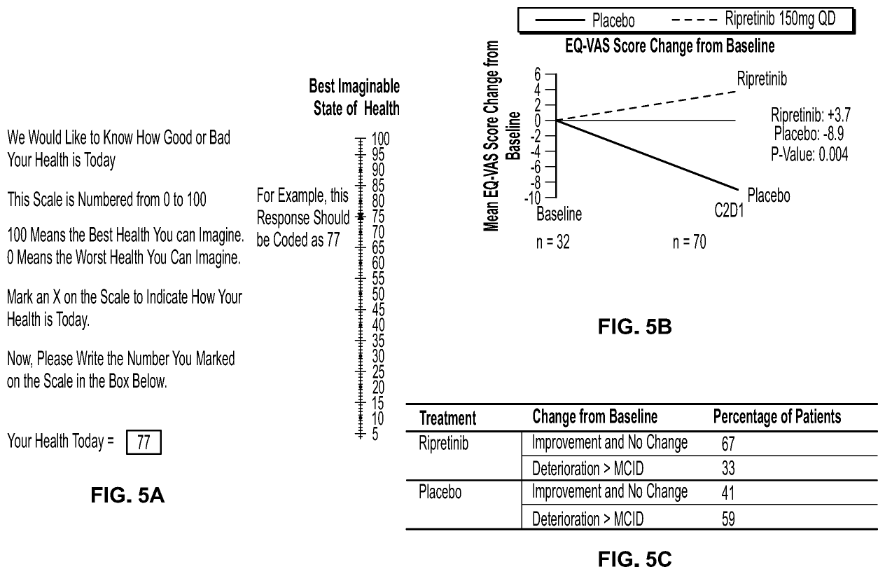

FIG. 5 depicts patient report outcome by EQ-VAS showing the visual scale used to assess scores (FIG. 5A), score changes from baseline (FIG. 5B) and corresponding patient percentage distributions (FIG. 5C). The EQ-VAS score improved on average 3.7 from baseline to Cycle 2, Day 1 (C2D1) among patients taking ripretinib in contrast to placebo patients who saw on average, a decline from baseline to C2D1 of 8.9 (p=0.004). 70 patients were receiving ripretinib and 32 were receiving placebo.

The EORTC QLQ-C30 is an assessment of function and symptoms of therapy by cancer patient, and is not specific to any cancer. The EORTC QLQ-C30 is a 30-question plus one global health status, including 5 functional scales, 3 symptom scales and a global health status.

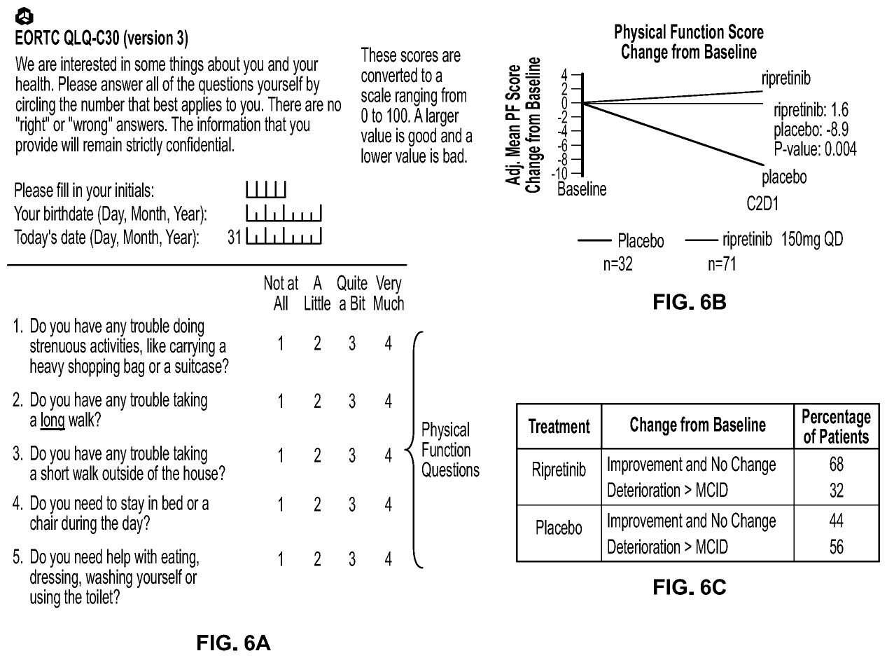

FIG. 6 depicts EORTC QLQ-C30 physical function questions (FIG. 6A), patient score changes from baseline in response (FIG. 6B) and corresponding patient percentage distributions (FIG. 6C). Physical function scores improved on average 1.6 from baseline to C2D1 among patients taking ripretinib in contrast to placebo patients who saw on average, a decline from baseline to C2D1 of 8.9 (p=0.004). 71 patients were receiving ripretinib and 32 were receiving placebo.

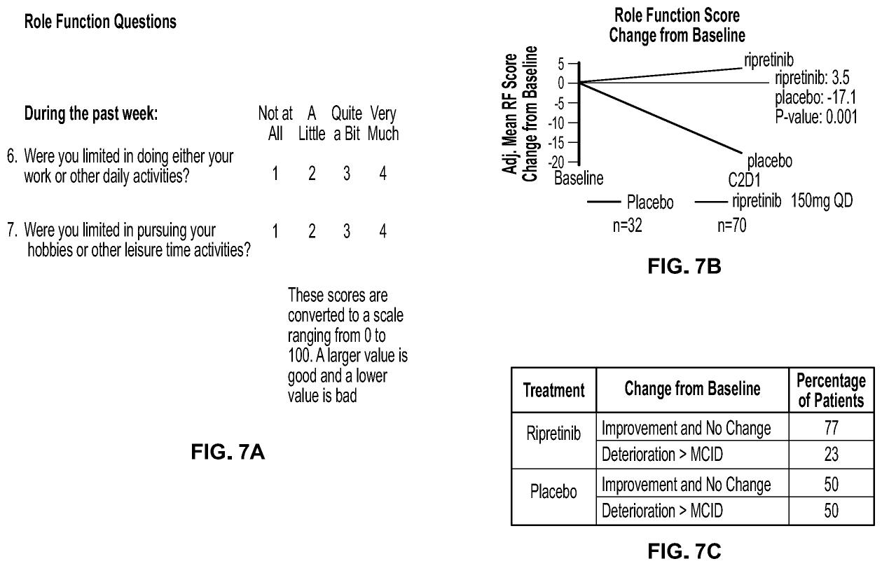

FIG. 7 depicts EORTC QLQ-C30 role function questions (FIG. 7A) and patient score changes from baseline in response (FIG. 7B) and corresponding patient percentage distributions (FIG. 7C). Role function score improved on average 3.5 from baseline to C2D1 among patients taking ripretinib in contrast to placebo patients who saw on average, a decline from baseline to C2D1 of 17.1 (p=0.001). 70 patients were receiving ripretinib and 32 were receiving placebo.

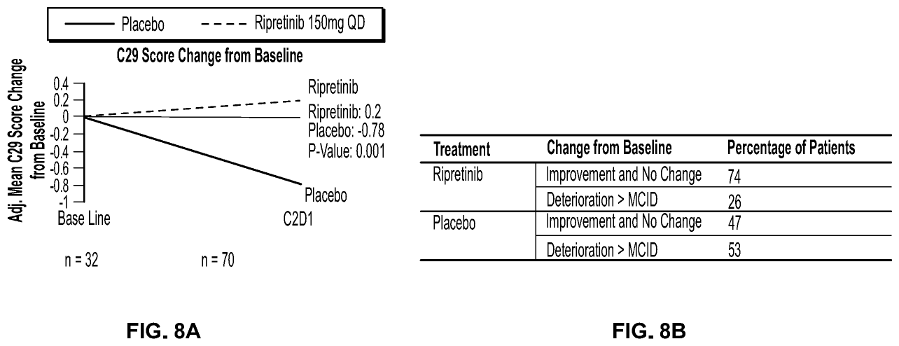

FIG. 8 depicts patient score changes from baseline (FIG. 8A) and corresponding patient percentage distributions (FIG. 8B) in response to Question C29 of EORTC QLQ-C30 (“How would you rate your overall health during the past week?”) from a scale of 1 (“Very poor”) to 7 (“Excellent”). There was a 0.20 improvement in C29 score in the ripretinib group compared to a 0.78 decrease in the placebo group (p=0.001). 70 patients were receiving ripretinib and 32 were receiving placebo.

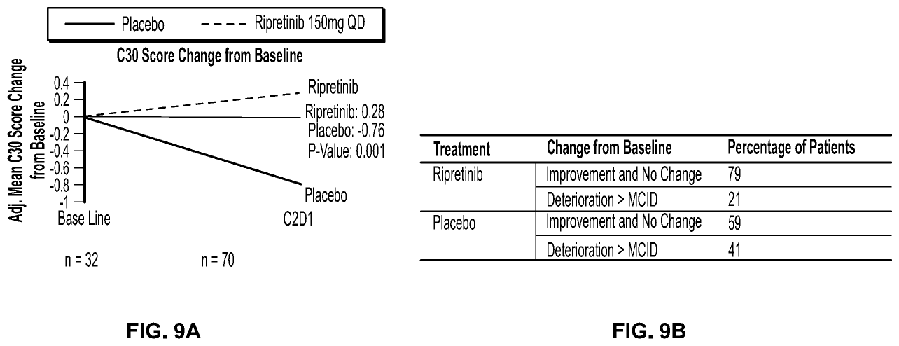

FIG. 9 depicts patient score changes from baseline (FIG. 9A) and corresponding patient percentage distributions (FIG. 9B) in response to Question C30 of EORTC QLQ-C30 (“How would you rate your overall quality of life during the past week?”) from a scale of 1 (“Very poor”) to 7 (“Excellent”). There was a 0.28 improvement in score in the ripretinib group compared to a 0.76 decrease in the placebo group (p=0.001). 70 patients were receiving ripretinib and 32 were receiving placebo.

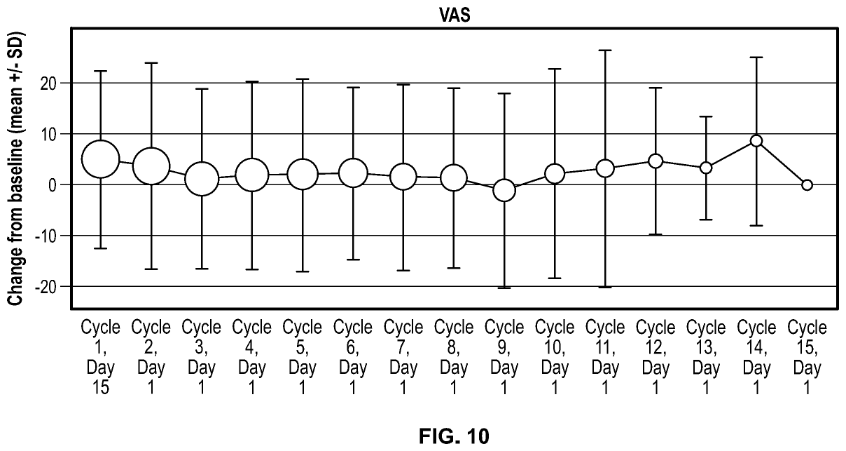

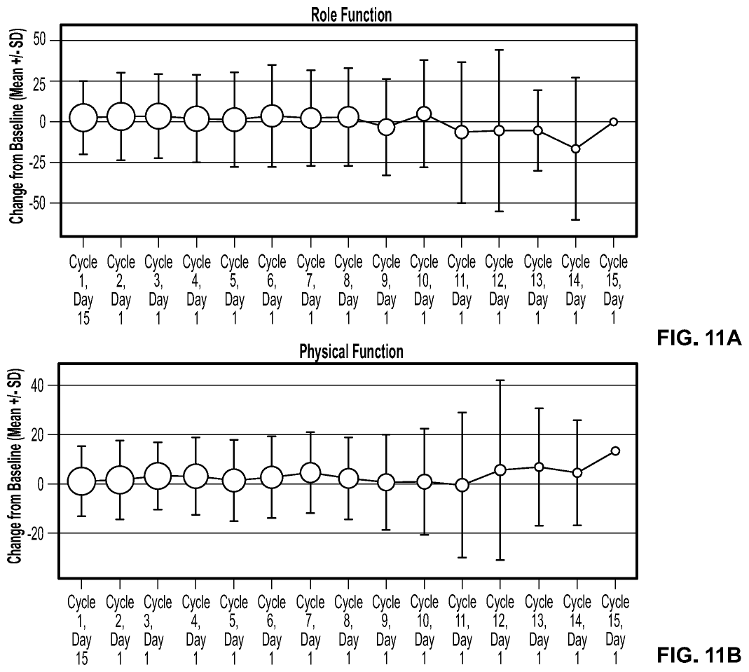

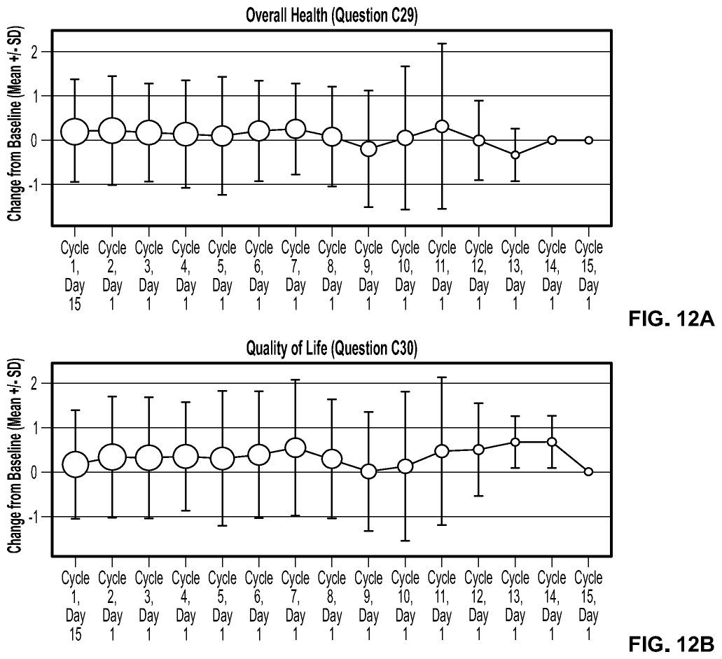

FIG. 10 depicts mean changes in baseline scores in EQ-VAS across various time points, from Cycle 1, Day 15 up to Cycle 15, Day 1 of the intention-to-treat population. FIG. 11A and FIG. 11B depict mean changes in baseline scores in EORTC QLQ-C30 role function and EORTC QLQ-C30 physical function, respectively, across various time points, from Cycle 1, Day 15 up to Cycle 15, Day 1 of the intention-to-treat population. FIG. 12A and FIG. 12B depict mean changes in baseline scores in EORTC QLQ-C30 question C29 response and EORTC QLQ-C30 question C30 response, respectively, across various time points, from Cycle 1, Day 15 up to Cycle 15, Day 1 of the intention-to-treat population.

### Open-Label Phase and Dose Escalation

Patients whose disease progressed during the double blind phase of the study were escalated to 150 mg BID ripretinib, continued at the 150 mg QD dose, or discontinued in an open label-phase of the study. Table 8 shows a comparison of patients that received 150 mg QD ripretinib at the data cutoff time point of the double blind phase and the data cutoff time point of of the open-label phase. The data in Table 8 suggest that data cut 9 months after the primary double-blind analysis has shown improvement in mOS and a similar mPFS in the ripretinib arm.

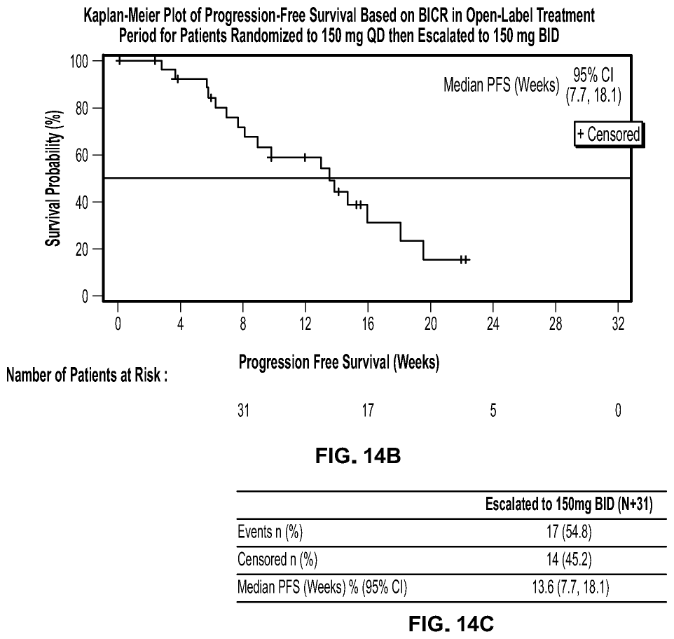

In the study, at least 31 patients dose escalated to 150 mg BID ripretinib in an open-label phase upon disease progression. PFS studies in the double-blind and open-label periods for these patients are depicted in FIG. 14A and FIG. 14B, respectively, with FIG. 14C depicting median PFS data.

### Wild Type (KIT and PDGFRA) Mutations

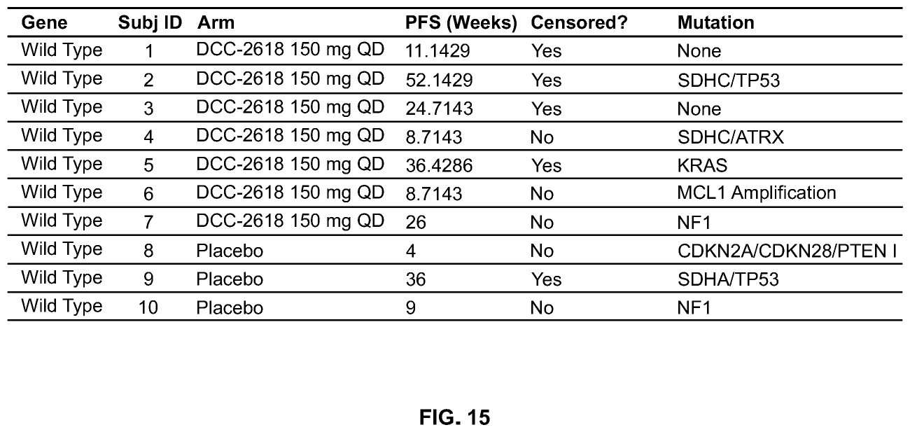

PFS data among patients with wild-type KIT or PDGFRA mutations were further evaluated at 150 mg ripretinib QD. PFS data of patients with these wild-type KIT and PDGFRA mutations are shown in FIG. 15.

### Additional Mutational Analyses

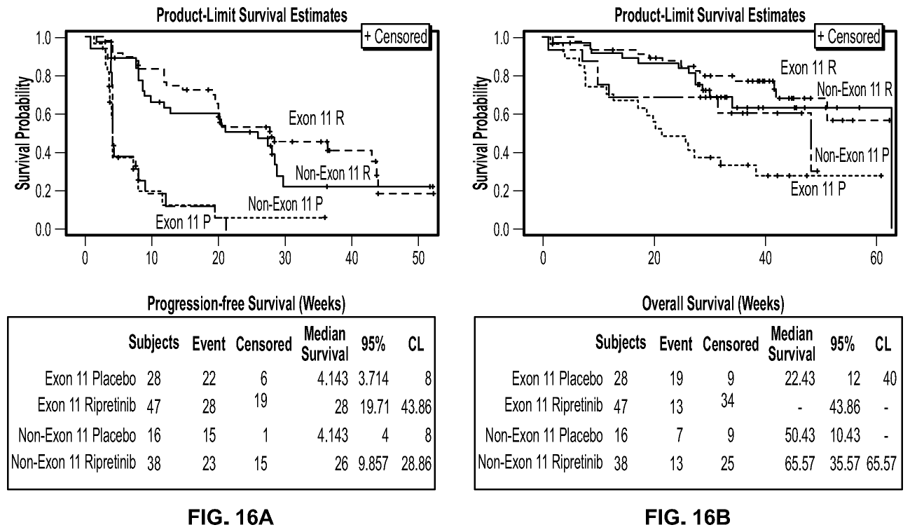

Progression free survival PFS and overall survival (OS) data based on patients with a primary Exon 11 mutation or patients with a non-Exon 11 mutation are shown in FIGS. 16A (PFS) and 16B (OS) at 150 mg ripretinib QD. The data show that, regardless of primary mutation, whether a primary Exon 11 or a non-Exon 11 primary mutation, GIST patients derive similar benefit from ripretinib over placebo.

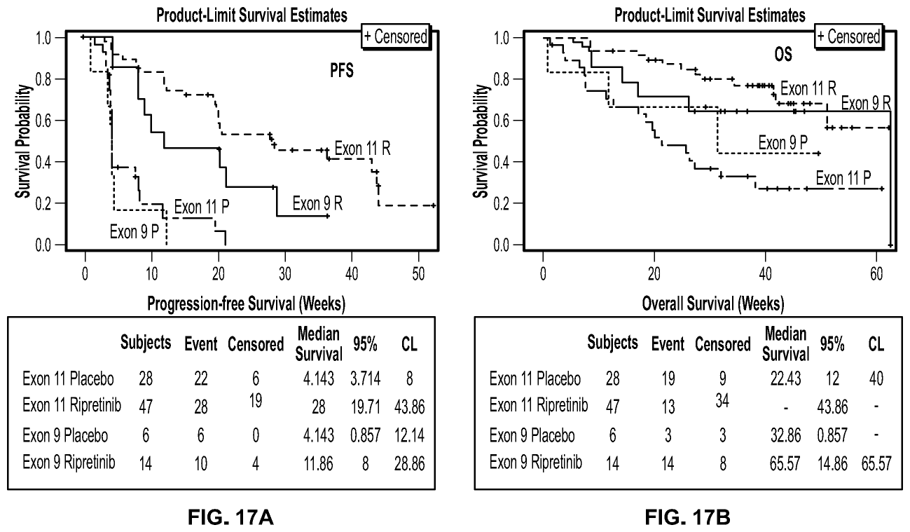

Progression free survival PFS and overall survival (OS) data based on patients with a primary Exon 11 mutation or those with a primary Exon 9 mutation are shown in FIGS. 17A (PFS) and 17B (OS) at 150 mg ripretinib QD. The data show that, Regardless of primary mutation, both Exon 11 and Exon 9 GIST patients derive a benefit from ripretinib over placebo.

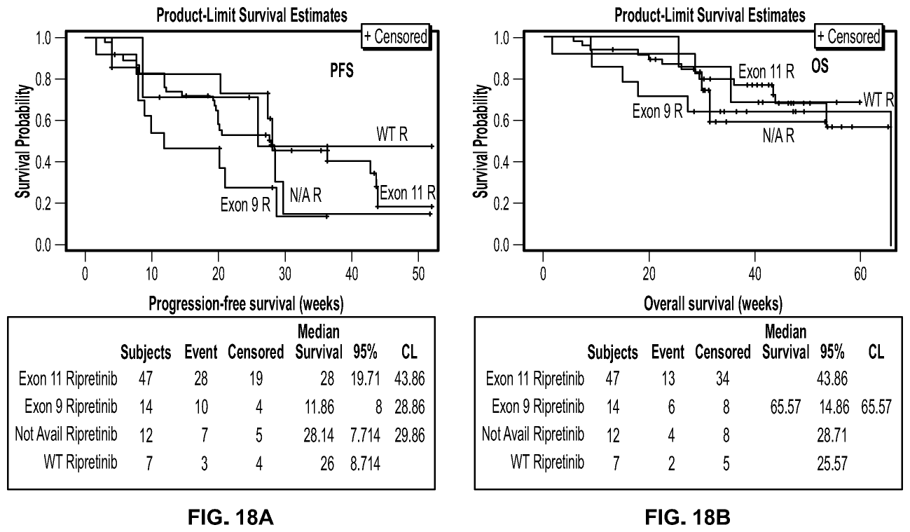

Furthermore, progression free survival PFS and overall survival (OS) data based on patients with a primary Exon 11 mutation, or a primary Exon 9 mutation, or other mutations, and wild type (KIT and PDGFRA) are shown in FIGS. 18A (PFS) and 19B (OS) at 150 mg ripretinib QD.

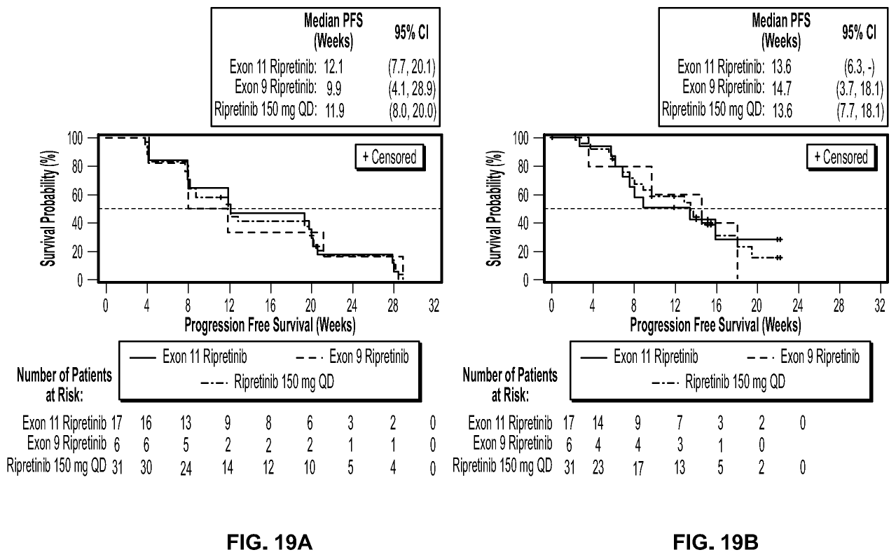

PFS studies for patients with certain primary mutations (Exon 9 or Exon 11) who dose escalated to 150 mg BID ripretinib are shown in FIGS. 19A and 19B for the double-blind and open-label periods, respectively.

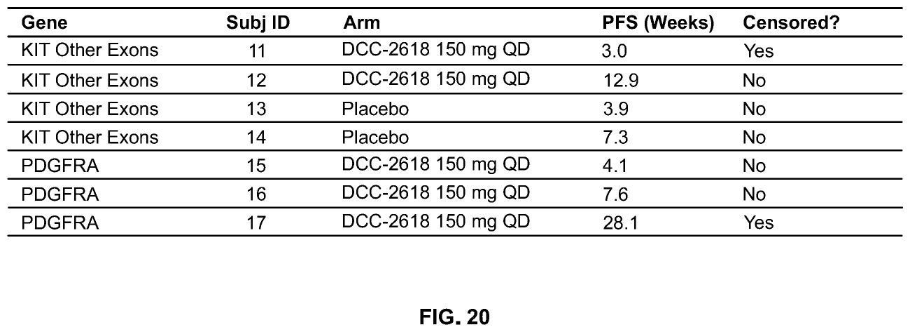

Additionally, FIG. 20 shows exemplary progression free survival data for patients with other KIT mutations and PGDFR mutations in the study of Example 1 at 150 mg ripretinib QD.

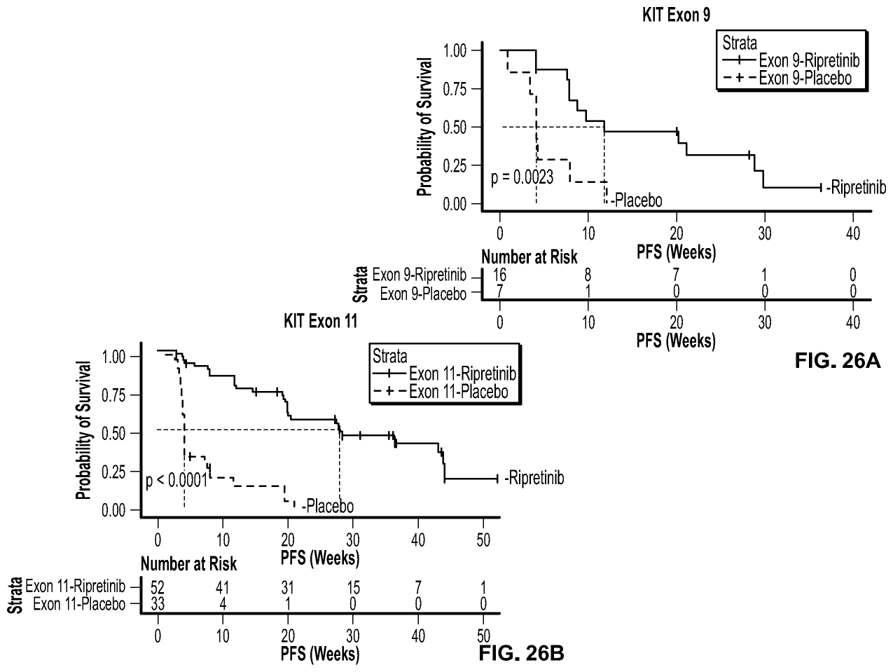

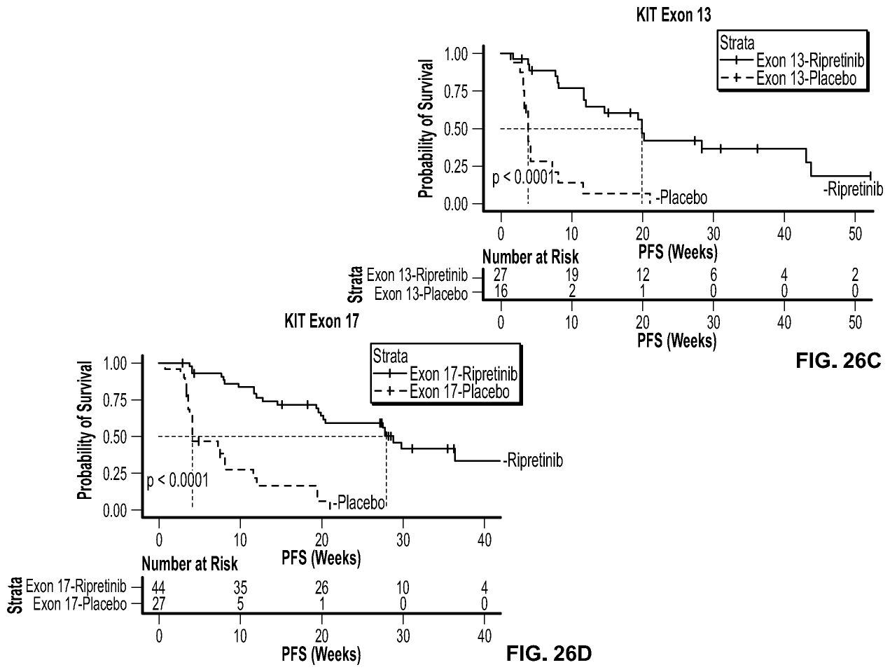

FIGS. 26A-D depicts an exemplary comparison of PFS of patient subgroups with Exon 9 (FIG. 26A), Exon 11 (FIG. 26B), Exon 13 (FIG. 26C), or Exon 17 (FIG. 26D) KIT mutations. This exemplary data show that ripretinib showed PFS benefit in all assessed patient subgroups compared to placebo.

### Example 2. Results for a Clinical Study of Ripretinib in Patients with Second-Line Through Fourth-Line Plus GIST

Results. Data from 178 GIST patients receiving ripretinib at doses of ≥100 mg daily are noted in Table 2. The table includes investigator-assessed objective response rate (ORR) by best response, disease control rate (DCR) and median progression free survival (mPFS), all of which were determined by Response Evaluation Criteria in Solid Tumors (RECIST) version 1.1.

TABLE 2
Results/clinical study of ripretinib

| | Objective | | | | |
|---|---|---|---|---|---|
| | Response | | | | |
| | Rate | | | | |
| | by Best | | Median | | |
| | Response | Disease | Progres- | | |
| | Includes | Control | sion | Censored | |
| | Unconfirmed | Rate | Free | Patients | Mean |
| Line of | (Confirmed | at 3 | Survival | for | Treatment |
| Therapy⁽¹⁾ | Only) | Months | (mPFS) | mPFS | Duration⁽²⁾⁽³⁾ |
| Second- | 30% (22%) | 81% | 42 weeks | 38% | 43 weeks |
| Line | | | | | |
| (n = 37) | | | | | |
| Third- | 23% (13%) | 80% | 40 weeks | 32% | 48 weeks |
| Line | | | | | |
| (n = 31) | | | | | |
| Fourth- | 15% (8%) | 73% | 30 weeks | 30% | 49 weeks |
| Line | | | | | |
| (n = 60) | | | | | |
| ≥Fourth- | 11% (7%) | 66% | 24 weeks | 22% | 41 weeks |
| Line | | | | | |
| (n = 110)⁽⁴⁾ | | | | | |

In Table 2, (1) indicates overall number of patients (n=178) remains the same as prior data presented at ESMO 2018; based on additional data cleaning, one patient from each of 2^{nd} line and 4^{th}/≥4^{th} line were reclassified as 3^{rd} line patients; (2) refers to median treatment durations were: 2^{nd} line=44 weeks, 3^{rd} line=48 weeks, 4^{th} line=46 weeks and ≥4^{th} line=29 weeks; (3) refers to including 60 patients who elected for intra-patient dose escalation from 150 mg QD to 150 mg BID; and (4) refers to the number of patients including 60 patients from 4th line.

Ripretinib was generally well tolerated and the updated adverse events were consistent with previously presented Phase 1 data in patients with GIST. Grade 3 or 4 treatment-emergent adverse events (TEAEs) in >5% of patients were lipase increased (18%; n=33), anemia (11%; n=20), hypertension (7%; n=13) and abdominal pain (6%; n=11). 13% of patients (n=24) experienced TEAEs leading to study treatment discontinuation, 17% of patients (n=31) experienced TEAEs leading to dose reduction and 49% of patients (n=88) had TEAEs leading to study drug interruption. Table 3 lists TEAEs>10% for GIST patients treated with ≥100 mg of ripretinib daily.

TABLE 3
Treatment-emergent adverse events for patients administered
with 100 mg of ripretinib daily.
GIST PATIENTS @ ≥100 MG DAILY
Treatment Emergent Adverse Events (TEAEs) >10%

| | | | |
|---|---|---|---|
| Alopecia | 102 (57%) | 0 (0%) | 102 (57%) |
| Fatigue | 94 (53%) | 4 (2%) | 98 (55%) |
| Myalgia | 79 (44%) | 0 (0%) | 79 (44%) |
| Nausea | 77 (43%) | 1 (1%) | 78 (44%) |
| Palmar-plantar erythrodysaesthesia | 71 (40%) | 1 (1%) | 72 (40%) |
| syndrome | | | |
| Constipation | 67 (37%) | 0 (0%) | 67 (37%) |
| Decreased appetite | 60 (34%) | 2 (1%) | 62 (35%) |
| Diarrhea | 50 (28%) | 3 (2%) | 53 (30%) |
| Weight decreased | 51 (29%) | 1 (1%) | 52 (29%) |
| Lipase increased | 18 (10%) | 33 (18%) | 51 (29%) |
| Muscle spasms | 47 (26%) | 0 (0%) | 47 (26%) |
| Abdominal pain | 33 (18%) | 11 (6%) | 44 (25%) |
| Vomiting | 42 (24%) | 2 (1%) | 44 (25%) |
| Arthralgia | 40 (22%) | 0 (0%) | 40 (22%) |
| Anemia | 18 (10%) | 20 (11%) | 38 (21%) |
| Hypertension | 25 (14%) | 13 (7%) | 38 (21%) |
| Cough | 37 (21%) | 0 (0%) | 37 (21%) |
| Dry skin | 37 (21%) | 0 (0%) | 37 (21%) |
| Dyspnea | 32 (18%) | 4 (2%) | 36 (20%) |
| Headache | 33 (18%) | 1 (1%) | 34 (19%) |
| Back Pain | 30 (17%) | 2 (1%) | 32 (18%) |
| Dizziness | 29 (16%) | 0 (0%) | 29 (16%) |
| Rash | 27 (15%) | 0 (0%) | 27 (15%) |
| Hypokalaemia | 21 (12%) | 5 (3%) | 26 (15%) |
| Hypophosphataemia | 17 (10%) | 8 (5%) | 25 (14%) |
| Actinic keratosis | 25 (14%) | 0 (0%) | 25 (14%) |
| Blood bilirubin increase | 16 (9%) | 5 (3%) | 21 (12%) |
| Amylase increased | 19 (11%) | 2 (1%) | 21 (12%) |
| Insomnia | 21 (12%) | 0 (0%) | 21 (12%) |
| Seborrhoeic keratosis⁽²⁾ | 21 (12%) | 0 (0%) | 21 (12%) |
| Urinary tract infection | 16 (9%) | 4 (2%) | 20 (11%) |
| Dysgeusia | 20 (11%) | 0 (0%) | 20 (11%) |
| Pain in extremity | 18 (10%) | 1 (1%) | 19 (11%) |
| Blood creatine phosphokinase | 13 (7%) | 5 (3%) | 18 (10%) |
| increased | | | |
| Upper respiratory tract infection | 18 (10%) | 0 (0%) | 18 (10%) |
| Rash maculo-papular | 18 (10%) | 0 (0%) | 18 (10%) |
| Hypomagnesaemia | 18 (10%) | 0 (0%) | 18 (10%) |
| Pruritus | 18 (10%) | 0 (0%) | 18 (10%) |
| Skin papilloma⁽²⁾ | 17 (10%) | 0 (0%) | 17 (10%) |
| Vision blurred | 17 (10%) | 0 (0%) | 17 (10%) |

In table 3, (1) refers to including one patient that only participated in the food effect portion of the Phase 1 study; and (2) indicates that dermatology skin exams were implemented to better evaluate skin lesions.

### Example 3. Results for a Clinical Study of 150 mg QD Ripretinib Administered to Patients with Second-Line Through Fourth-Line Plus GIST

Efficacy and safety results from the escalation and expansion phases of a phase 1 study for patients with GIST treated at ripretinib 150 mg QD as the starting dose in 28-day cycles are presented. Local, investigator-assessed Response Evaluation Criteria in Solid Tumors (RECIST 1.1) response assessments were performed every 2 cycles, and patients in the expansion cohorts who progressed per RECIST 1.1 were allowed to dose escalate to 150 mg BID.

142 patients with GIST in the escalation and expansion phases were treated at 150 mg QD dose. Number of patients by line of therapy were as follows: 31 2^{nd} line, 28 3^{rd} line, and 83≥4^{th} line patients. 135 patients (95.1%) had KIT-mutant GIST, and 7 patients (4.9%) had PDGFRA-mutant GIST.

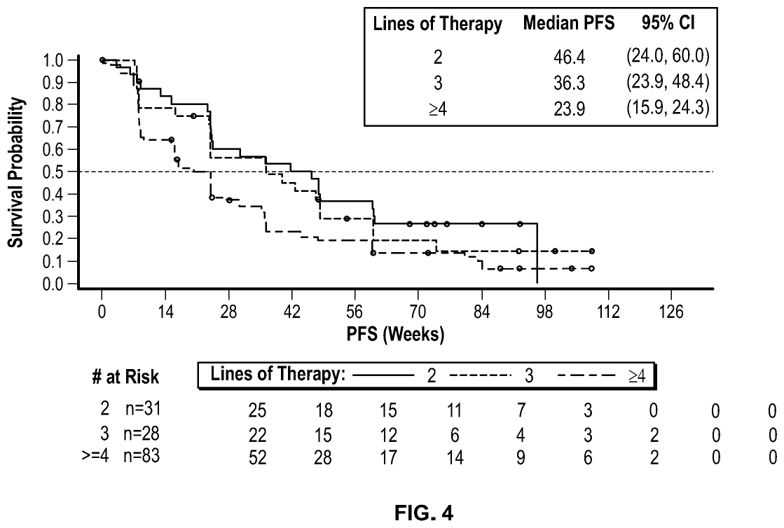

Results on the efficacy by line of therapy in patients with GIST receiving ripretinib 150 mg QD are presented in Table 4. For example, the confirmed-only complete response (CR), partial response (PR), stable disease, and progressive disease are presented in Table 4. The objective response rate data in Table 4 relates to the proportion of patients with CR+PR. The median PFS data in Table 4 refers to progression-free survival per investigator assessment, by line of therapy. PFS plots by line of therapy are also shown in FIG. 4.

TABLE 4
Efficacy by line of therapy in patients with GIST
receiving ripretinib 150 mg QD.

| | 2^{nd} Line | 3^{rd} Line | ≥4^{th} Line |
|---|---|---|---|
| Parameters | (n = 31) | (n = 28) | (n = 83) |
| Best response (confirmed | | | |
| only), n (%) | | | |
| CR | 0 | 0 | 0 |
| PR | 6 (19.4) | 4 (14.3) | 6 (7.2) |
| Stable disease | 21 (67.7) | 18 (64.3) | 49 (59.0) |
| Progressive disease | 4 (12.9) | 6 (21.4) | 22 (26.5) |
| Not evaluable | 0 | 0 | 1 (1.2) |
| No response assessment | 0 | 0 | 5 (6.0) |
| ORR, n (95% CI) | 19.4 (7.5, 37.5) | 14.3 | 7.2 (2.7, 15.1) |
| | | (4.0, 32.7) | |
| Duration of treatment^{a} | | | |
| Mean, weeks (SD) | 56.1 (34.24) | 57.5 (32.95) | 44.9 (36.58) |
| Median, weeks | 64 | 51 | 29 |
| Min, Max | 4, 132 | 8, 124 | 0.1, 140 |
| Duration of response | | | |
| n | 6 | 4 | 6 |
| Number of events | 3 | 1 | 3 |
| Median, weeks | 80 | NE | 76.1 |
| 95% CI | 24.7, 80.0 | 52.1, NE | 24.1, NE |
| PFS | | | |
| Number of censored | 8 | 6 | 12 |
| patients | | | |
| Median, weeks | 46.4 | 36.3 | 23.9 |
| 95% CI | 24.0, 60.0 | 23.9, 48.4 | 15.9, 24.3 |

In Table 4: ^{a}64 subjects escalated to 150 mg BID among patients with GIST in the 150 mg QD dose group. CI, confidence interval; CR, complete response; NE, not estimable; ORR, objective response rate; PFS, progression-free survival; PR, partial response; SD, standard deviation. Local (investigator) response assessment.

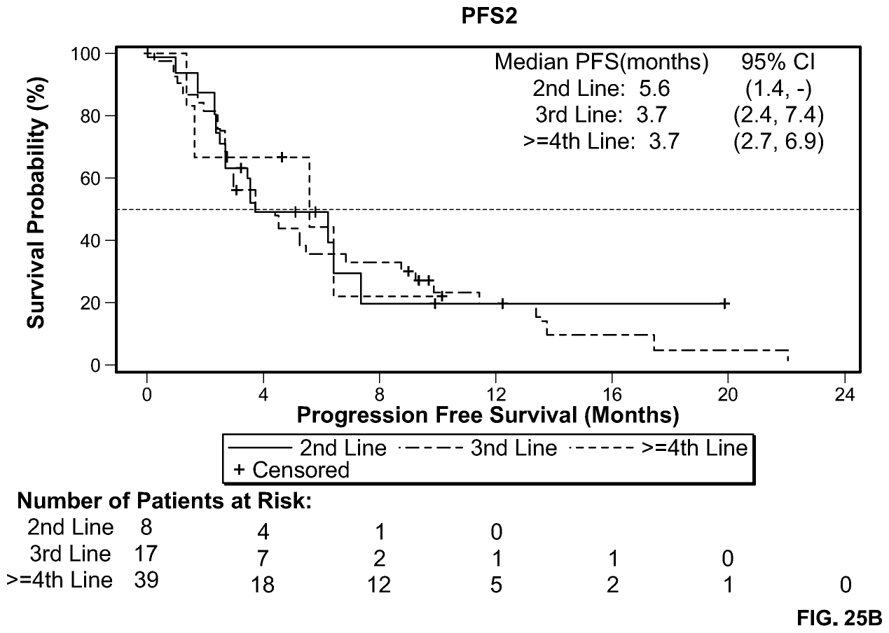

In subjects dose escalated to 150 mg BID, PFS before (PFS1) and after (PFS2) dose escalation was evaluated and is shown in FIGS. 25A and 25B, respectively. The data support that, regardless of line of therapy, patients received additional clinical benefit by investigator assessment after dose escalation to 150 mg BID.

### Mutational Analysis

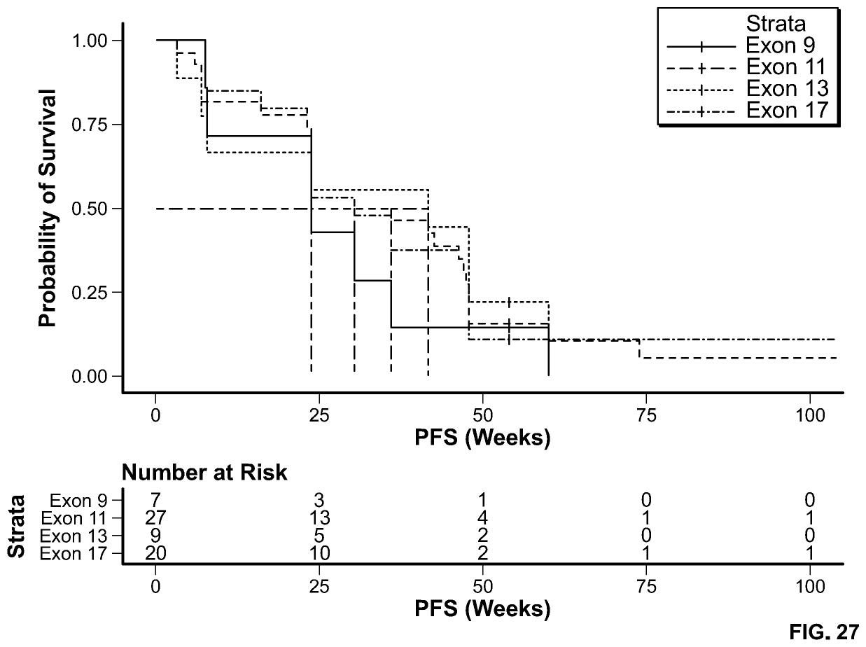

FIG. 27 shows PFS data of exemplary second and third-line patients with Exons 9, 11, 13, or 17 KIT mutations. The data show that, in second and third line populations, patients with such various KIT mutations have comparable PFS.

### Example 4. Protocol for Dose Modification of Ripretinib as a Result of Adverse Reactions

If dose modifications of ripretinib are required due to adverse reactions, the following protocol will be applied: reduce the dose in 50 mg (one tablet) increments; the lowest recommended dose of ripretinib is 50 mg once daily. Ripretinib dosage reductions for adverse reactions are summarized in Table 5.

TABLE 5
Recommended Dose Reduction for Adverse Reactions

| Dose Level | Dose |
|---|---|
| Recommended starting dose | 150 mg once daily |
| First dose reduction | Reduce to 100 mg once daily |
| Second dose reduction | Reduce to 50 mg once daily |
| (lowest recommended dose) | |

Dosing will be reduced, interrupted, or discontinued for certain toxicities. See Table 6 for dose modification guidelines.

TABLE 6
Recommended Dose Modifications for ripretinib

| Adverse | | |
|---|---|---|
| Reaction | Severity^{a} | Dosage Modifications |
| Hand-foot | Grade 2 | Interrupt ripretinib for at least 7 days. |
| skin reaction | | If the event returns to Grade 1 or baseline |
| (HFSR) | | within 7 days, resume ripretinib at the same |
| [palmar- | | dose level. |
| plantar | | If the event returns to Grade 1 or baseline |
| erythro- | | after 7 days, resume ripretinib at a reduced |
| dysesthesia | | dose level (see Table 5). |
| syndrome | | If after a dose reduction, the event is |
| (PPES)] | | maintained at Grade 1 or baseline for at |
| | | least 28 days, consider re-escalating |
| | | ripretinib by 1 dose level (see Table 5). |
| | | If this is a recurrence, after event returns to |
| | | Grade 1 or baseline, resume ripretinib at a |
| | | reduced dose level (see Table 5) regardless |
| | | of time to improvement. |
| | Grade 3 | Interrupt ripretinib for at least 7 days or |
| | | until the event returns to Grade 1 or |
| | | baseline (maximum 28 days). Resume |
| | | ripretinib at a reduced dose level (see Table |
| | | 5). |
| | | If after a dose reduction the event is |
| | | maintained at Grade 1 or baseline for at |
| | | least 28 days of dosing, consider re- |
| | | escalating ripretinib by 1 dose level (see |
| | | Table 5). |
| Hyper- | Grade 3 | Medically manage hypertension to achieve |
| tension | | normal blood pressure. |
| | | If symptomatic hypertension, withhold |
| | | ripretinib and treat hypertension. Resume |
| | | ripretinib at the same dose level after |
| | | symptoms have resolved. |
| | | If blood pressure is not controlled with |
| | | medical management, reduce ripretinib to |
| | | the next dose level (see Table 5). |
| | | If Grade 3 hypertension recurs despite |
| | | ripretinib dose reduction and medical |
| | | management, reduce ripretinib to the |
| | | lowest recommended dose of 50 mg once |
| | | daily. |
| | Grade 4 | Discontinue ripretinib permanently. |
| | Life- | |
| | threatening | |
| | consequences | |
| | (e.g., | |
| | malignant | |
| | hypertension, | |
| | transient or | |
| | permanent | |
| | neurologic | |
| | deficit, | |
| | hypertensive | |
| | crisis) | |
| Arthralgia/ | Grade 2 | Interrupt ripretinib for at least 7 days. |
| Myalgia | | If the event returns to Grade 1 or baseline |
| | | within 7 days, resume ripretinib at the same |
| | | dose level. |
| | | If the event returns to Grade 1 or baseline |
| | | after 7 days, resume ripretinib at a reduced |
| | | dose level (see Table 5). |
| | | If after a dose reduction, the event is |
| | | maintained at Grade 1 or baseline for at |
| | | least 28 days of dosing, consider re- |
| | | escalating ripretinib by 1 dose level (see |
| | | Table 5). |
| | | If this is a recurrence, after event returns to |
| | | Grade 1 or baseline, resume ripretinib at a |
| | | reduced dose level (see Table 5) regardless |
| | | of time to improvement. |
| | Grade 3 | Interrupt ripretinib for at least 7 days or |
| | | until the event returns to Grade 1 or |
| | | baseline (maximum 28 days). Resume |
| | | ripretinib at a reduced dose level (see Table |
| | | 5). |
| | | If after a dose reduction the event is |
| | | maintained at Grade 1 or baseline for at |
| | | least 28 days of dosing, consider re- |
| | | escalating ripretinib by 1 dose level (see |
| | | Table 5). |
| Other | Grade 3 or | Interrupt ripretinib until toxicity resolves to |
| adverse | higher | Grade 1 or baseline (maximum 28 days). |
| reactions | toxicity | If the event returns to Grade 1 or baseline, |
| | considered | resume ripretinib at a reduced dose level |
| | related to | (see Table 5). |
| | treatment | If the reduced dose is tolerated without |
| | | recurrence of the event for at least 28 days, |
| | | consider re-escalating ripretinib to the prior |
| | | dose level (see Table 5). |
| | | If Grade 3 or higher toxicity recurs, |
| | | discontinue ripretinib. |

BP = blood pressure;
DBP = diastolic blood pressure;
SBP = systolic blood pressure
^{a}Graded per National Cancer Institute Common Terminology Criteria for Adverse Events (NCI CTCAE) version 4.03

### Example 5. Safety of Ripretinib and Impact of Alopecia and Palmar-Plantar Erythrodysesthesia (PPES) on Patient-Reported Outcomes

The safety of ripretinib and the impact of alopecia and palmar-plantar erythrodysesthesia (PPES) on patient-reported outcomes (PROs) of patients treated in the study described in Example 1 herein are described. Ripretinib had a favorable overall safety and tolerability profile in the trial of Example 1. When stratified by alopecia and PPES, patient reported assessments of function, overall health, and overall quality of life were maintained over time. For both alopecia and PPES, onset and maximum severity occurred almost simultaneously, indicating that these events generally did not progressively worsen. Overall, these results suggest that alopecia and PPES are manageable and that ripretinib treatment offsets any negative impact associated with these AEs.

Patient reported outcomes (PROs) were assessed with questions from the EuroQol 5D (EQ 5D 5L) and the European Organization for the Research and Treatment of Cancer Quality of Life Questionnaire (EORTC QLQ C30).

Generalized estimating equation (GEE) models were used in statistical analyses in which: Repeated measures models across visits where the outcome was 1 of the 5 PROs; models were built only for ripretinib patients; for alopecia patients, cycles 1 and 2 were excluded to account for median time of alopecia onset; covariates were sex, alopecia/PPES (yes/no), and Eastern Cooperative Oncology Group (ECOG) score at baseline; and when there was no end date for the AE, it was coded conservatively as having extended to the last visit of the double blind period.

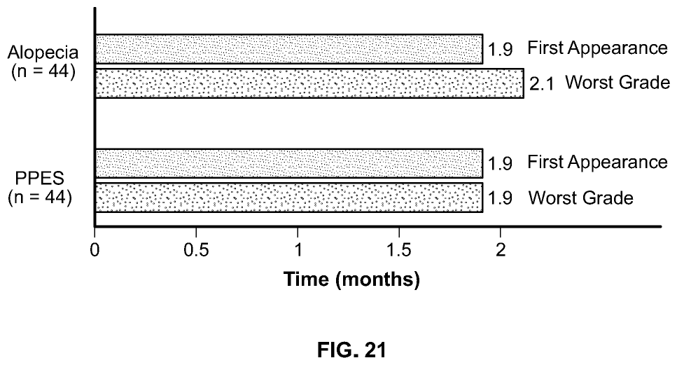

In the ripretinib arm, the most common treatment-emergent adverse event (TEAE) was alopecia (51.8%) and the most common grade 3/4 TEAE was anemia (9.4%). The highest severity classification for alopecia is grade 2; therefore, no patients in either arm had grade 3/4 alopecia. Alopecia was slightly more common in femalesvs males in the ripretinib arm (56.8% vs 43.2%). In the ripretinib arm, 21.2% of patients reported PPES; no patients had grade 3 PPES (grade 3 is the highest severity classification for PPES). There were no serious adverse events of alopecia or PPES reported. Within the ripretinib arm, 7.1%, 23.5%, and 5.9% of patients experienced a TEAE leading to dose reduction, dose interruption, or death, respectively, compared with 2.3%, 20.9%, and 23.3% in the placebo arm. In patients receiving ripretinib, the median worst grade of alopecia occurred very shortly after the median first appearance (FIG. 21). The median first appearance and worst grade of PPES occurred simultaneously in patients receiving ripretinib (FIG. 21).

Table 7 shows a GEE analysis summary of the association between alopecia and PPES with the 5 PRO measures in patients taking ripretinib. In a repeated measures analysis, there was a trend toward an improvement of the 5 PROs among patients with alopecia (Table 7). The presence of alopecia was associated with better self-reported overall quality of life (Table 7). This was statistically significant at P<0.01, but did not exceed the threshold for meaningful change. There was no association between PPES and the 5 PRO measures (Table 7).

TABLE 7
GEE analysis summary of the association between alopecia and
PPES with the 5 PRO measures in patients taking ripretinib.

| | | |
|---|---|---|
| Mean | Mean Confidence | P-value |
| Estimate | Limit | from ChiSq |

Alopecia
EORTC-QLQ-C30

| | | | |
|---|---|---|---|
| Overall health | 0.17 | (−0.10, 0.44) | 0.2222 |
| Overall quality of life | 0.35 | (0.03, 0.67) | 0.0313 |
| Physical function | 3.17 | (−0.29, 6.64) | 0.0729 |
| Role function | 4.50 | (−2.87, 11.87) | 0.2310 |

EQ-5D-5L

| | | | |
|---|---|---|---|
| VAS | 3.01 | (−0.64, 6.67) | 0.1062 |

PPES
EORTC-QLQ-C30

| | | | |
|---|---|---|---|
| Overall health | 0.06 | (−0.29, 0.41) | 0.7457 |
| Overall quality of life | 0.12 | (−0.26, 0.50) | 0.5368 |
| Physical function | 3.03 | (−0.92, 6.99) | 0.1325 |
| Role function | 2.83 | (−5.52, 11.17) | 0.5070 |

EQ-5D-5L

| | | | |
|---|---|---|---|
| VAS | 1.65 | (−2.11, 5.41) | 0.3903 |

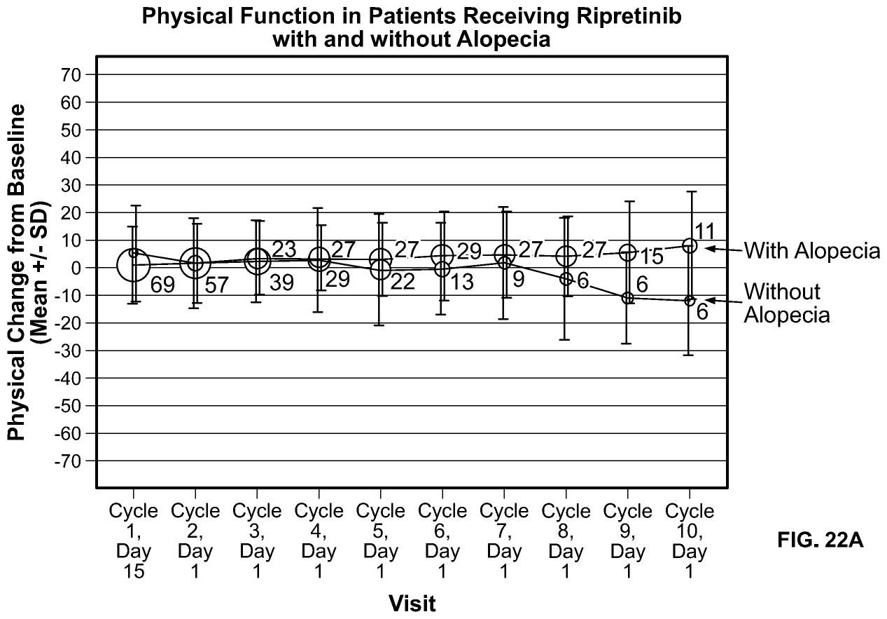

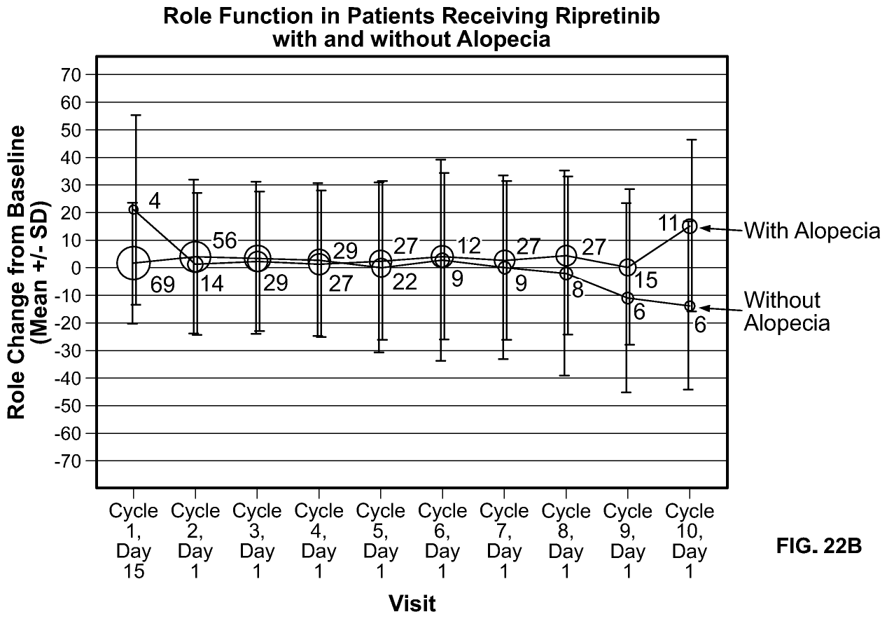

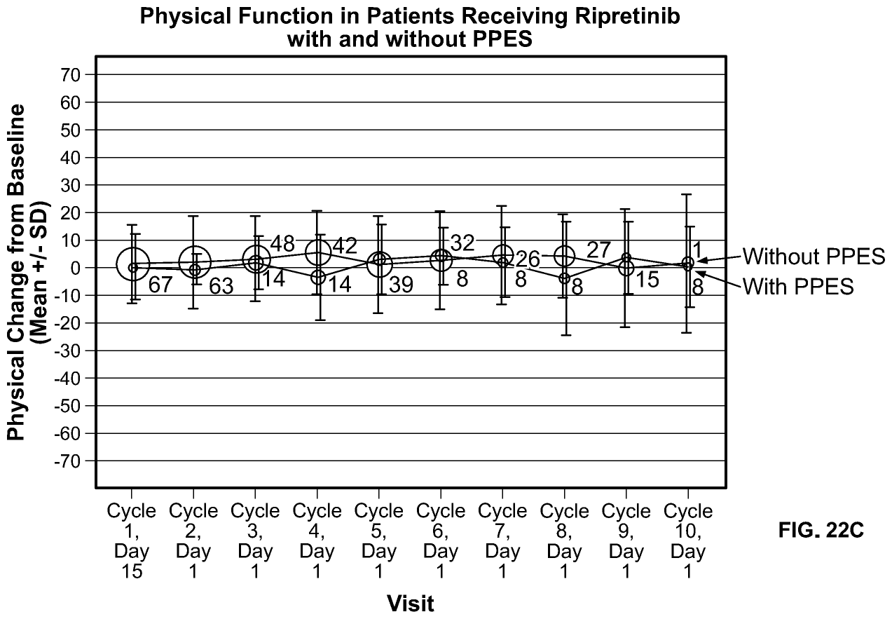

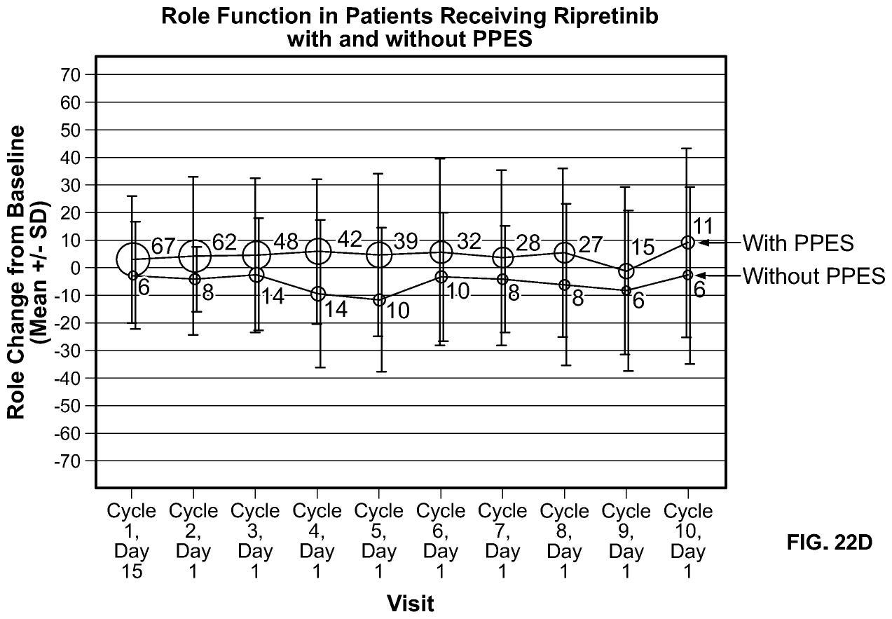

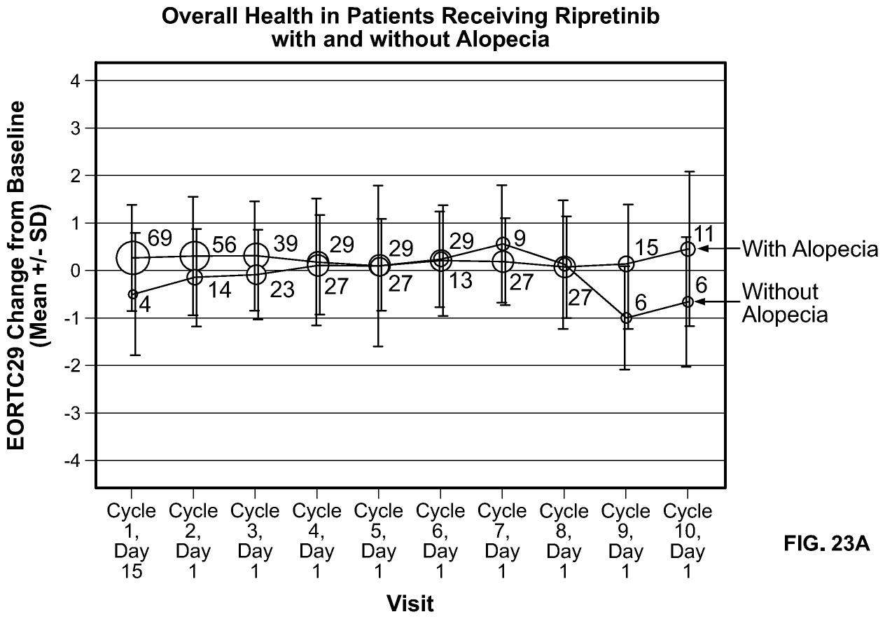

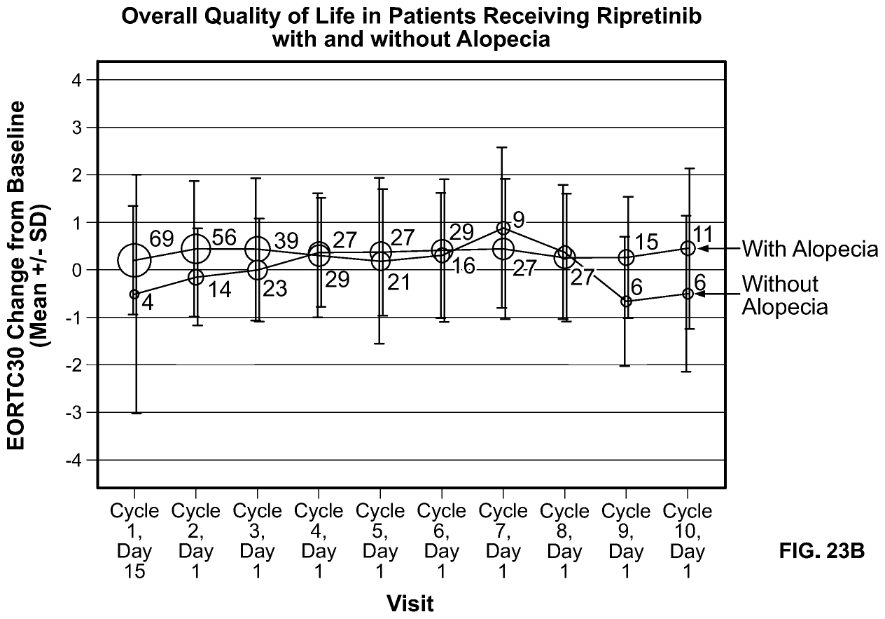

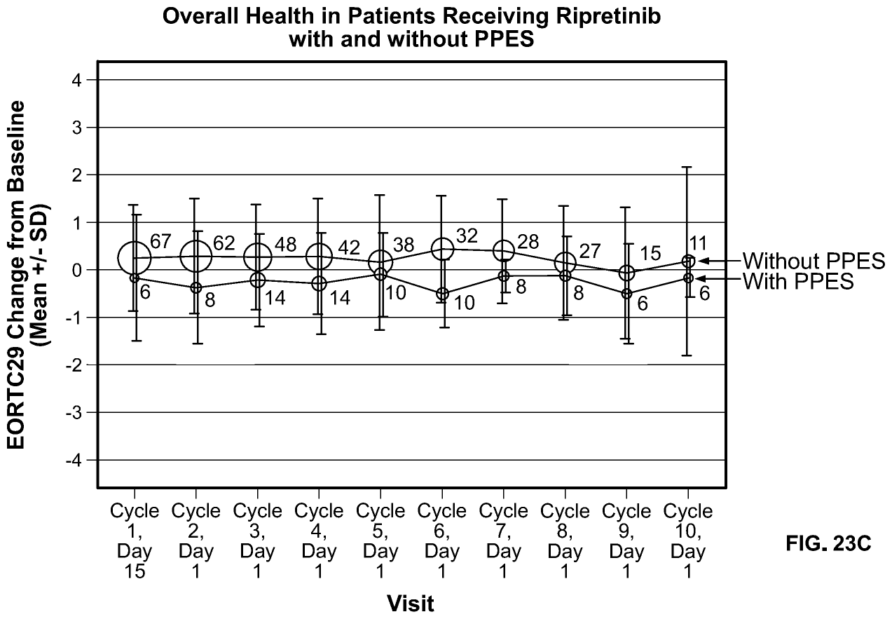

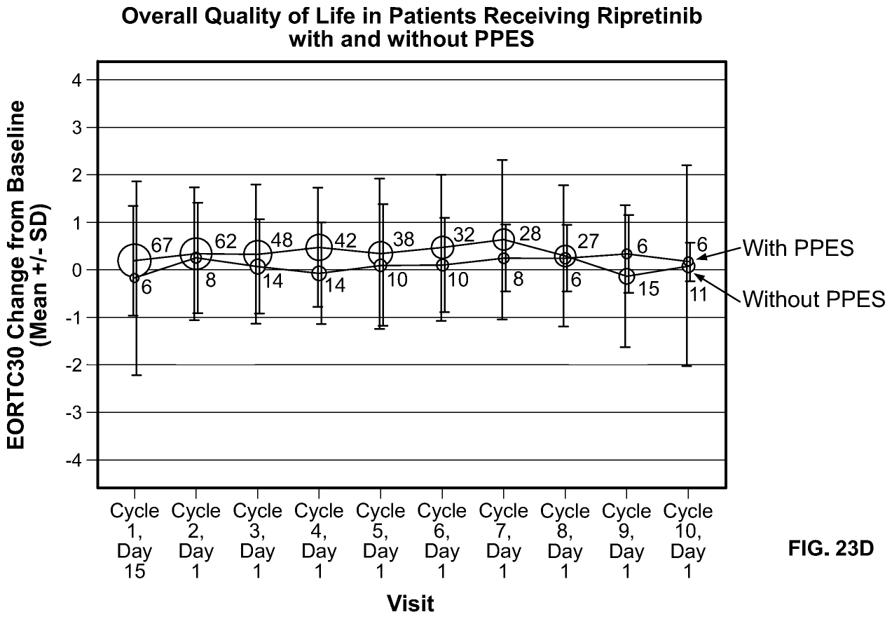

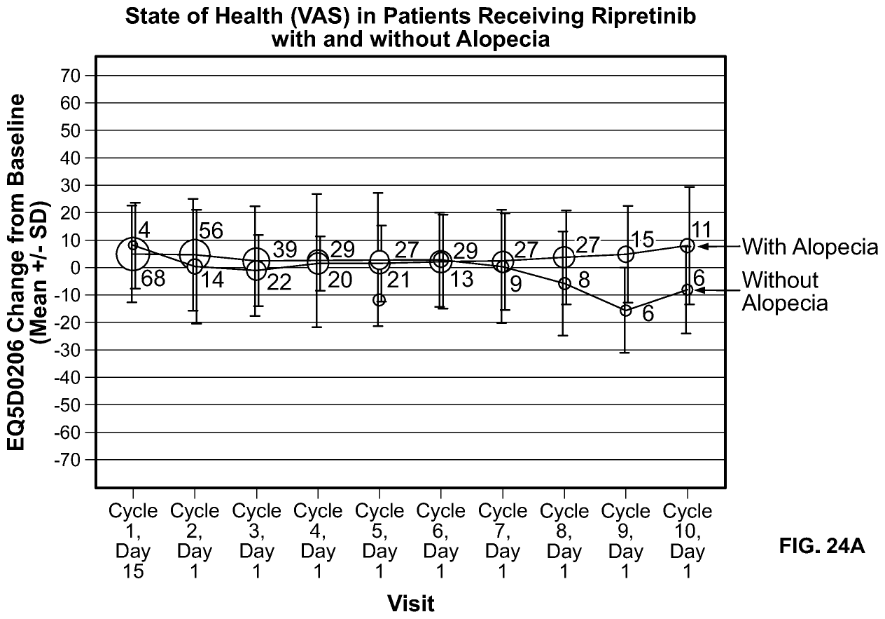

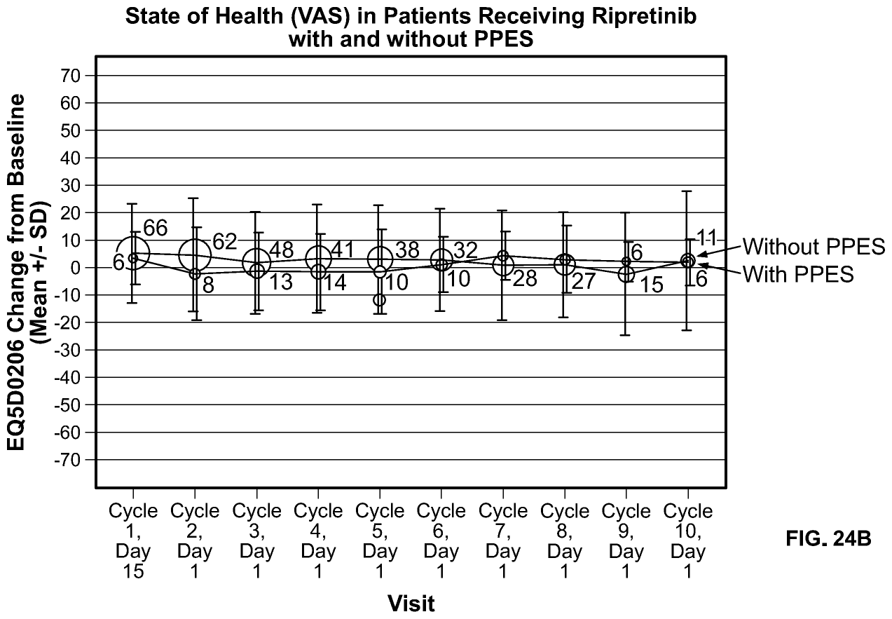

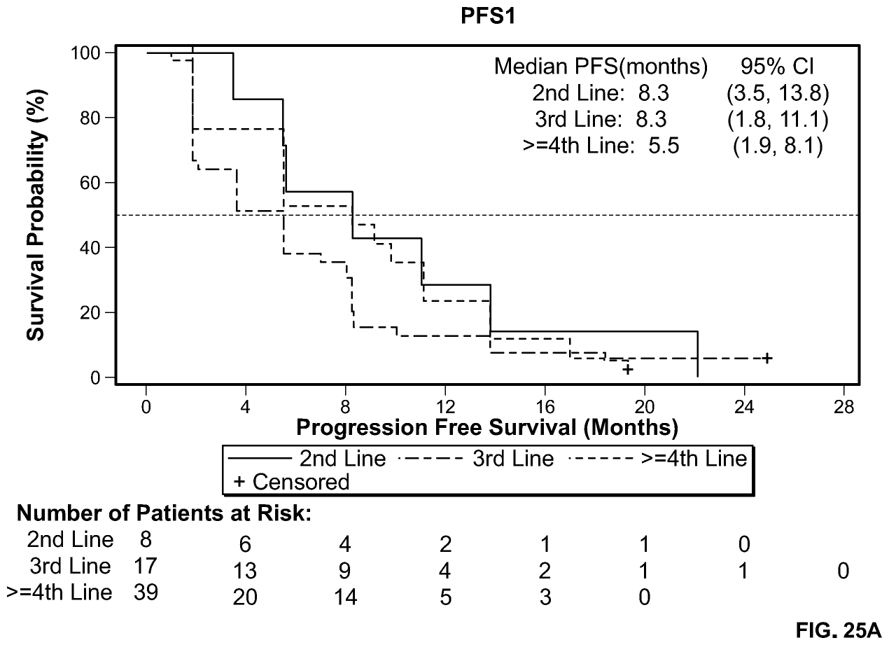

Longitudinal graphs out to Cycle 10, Day 1 demonstrate similar trends in mean change from baseline for the 5 PROs for patients receiving ripretinib that developed alopecia or PPES and those that did not (FIGS. 22A, 22B, 22C, 22D, 23A, 23B, 23C, 23D, 24A, and 24B).

TABLE 8
Comparison of double blind and open label data cut time periods.

| | Ripretinib | Ripretinib |
|---|---|---|
| | (N = 85) | (N = 85) |
| | Double-blind cut | Open-label cut |
| Progression-Free Survival by | | |
| BICR | | |
| Number of events (%) | 51 (60) | 64 (75) |
| Progressive disease | 46 (54) | 58 (68) |
| Deaths | 5 (6) | 6 (7) |
| Median PFS (months) (95% CI) | 6.3 (4.6, 6.9) | 6.3 (4.6, 8.1) |
| Hazard ratio (95% CI) | 0.15 (0.09, 0.25) | 0.16 (0.10, 0.27) |
| p-value | <0.0001 | <0.0001* |
| Overall Response Rate by BICR | | |
| Overall Response Rate (%) | 9 | 12 |
| (95% CI) | (4.2, 18) | (5.8, 20.6) |
| p-value | 0.0504 | 0.0156* |
| Overall Survival | | |
| Number of deaths (%) | 26 (31) | 38 (45) |
| Median OS (months) (95% CI) | 15 (12, 15) | Not Reached |
| | | (13, NE) |
| Hazard ratio (95% CI)/p-value | 0.36 (0.21,0.62)/ | 0.43 (0.26, 0.69)/ |
| | 0.0004* | 0.0014* |
| Exposure | | |
| Mean in the double blind period | 5.6 | 7.6 |
| (months) | | |

### Example 6. Studies of Ripretinib and Compound A with Strong CYP3A Inhibitors

Coadministration of 150 mg QD ripretinib with a strong CYP3A inhibitor increased the exposure of ripretinib and its active metabolite (Compound A), which may increase the risk of adverse reactions. Coadministration of ripretinib with itraconazole (a strong CYP3A inhibitor and also a P-gp inhibitor) increased ripretinib Cₘₐₓ by 36% and AUC_{0-inf} by 99% and also increased Compound A AUC_{0-inf} by 99% with no change in its Cₘₐₓ.

### Example 7. Studies of Ripretinib with a Proton-Pump Inhibitor

The effect of a proton-pump inhibitor on the exposure of ripretinib was evaluated. No clinically significant differences in the plasma exposure to ripretinib and Compound A were observed when ripretinib was coadministered with pantoprazole, a proton-pump inhibitor. Although ripretinib has pH-dependent solubility, concomitant administration of 40 mg QD pantoprazole with 150 mg QD ripretinib did not affect ripretinib exposure.

### Example 8. Studies of Food Effect on Ripretinib and Compound A Exposure

The effect of a high-fat breakfast on ripretinib and Compound A exposure was evaluated. A high fat meal consisted of approximately 150, 250, and 500-600 calories from protein, carbohydrate, and fat, respectively. Following administration of ripretinib with a high-fat meal at a 150 mg dose, ripretinib AUC_{0-24 h} and Cₘₐₓ were higher by 30% and 22%, respectively. For the metabolite Compound A, AUC_{0-24 h} and Cₘₐₓ were higher by 47% and 66%, respectively. The food effect is not considered to be clinically significant based on exposure-response analysis. Therefore, ripretinib may be taken with or without food at approximately same time each day.

### Example 9. Clinical Study of Ripretinib Treatment in a Patient Having a Pre-Existing Child-Pugh Class C Severe Hepatic Impairment and Suffering from Advanced Gastrointestinal Stromal Tumor

The effects of ripretinib in patients suffering from advanced gastrointestinal stromal tumors as well as a pre-existing Child-Pugh class C severe hepatic impairment was evaluated in a Phase 1, open-label study. Patients having severe hepatic impairment are compared to matched healthy subjects with no hepatic impairment. All patients are administered a 50 mg dose of ripretinib once daily.

Patients having severe hepatic impairment had a ripretinib AUC₀₋ₜ of about 160% higher or about 163% higher compared to a ripretinib AUC₀₋ₜ of a healthy subjects with no hepatic impairment. Patients with a severe hepatic impairment had a ripretinib (max of about 24% lower compared to a ripretinib (max of a healthy subject with no hepatic impairment.

### Example 10. Pharmacokinetic Studies of Ripretinib and its Active Metabolite

A summary of PK parameter results based on Child-Pugh Classification from the study of Example 9 is provided herein. PK parameters for ripretinib were as follows: For participants with mild hepatic impairment (HI) and their healthy matches: AUC₀₋ₜ (h×ng/ml) mean (CV %) was 3845 (53.8) and 3241 (46.1), respectively, with a ratio (geometric least squares [LS] mean [90% CI]) of 110 (69.2-176); AUC_{0-∞} (h×ng/ml) mean (CV %) was 3949 (52.7) and 3306 (45.4), respectively, with a ratio (geometric LS mean [90% CI]) of 111 (70.6-176); and Cₘₐₓ (ng/ml) mean (CV %) was 229.5 (37.0) and 284.3 (52.0), respectively, with a ratio (geometric LS mean [90% CI]) of 83.6 (56.7-123). For participants with moderate HI and their healthy matches: AUC₀₋ₜ (h×ng/ml) mean (CV %) was 6957 (71.5) and 3195 (51.9), respectively, with a ratio (geometric LS mean [90% CI]) of 199 (117-339); AUC_{0-∞} (h×ng/ml) mean (CV %) was 7131 (70.9) and 3275 (51.1), respectively, with a ratio (geometric LS mean [90% CI]) of 199 (118-337); Cₘₐₓ (ng/ml) mean (CV %) was 288.4 (48.8) and 294.0 (53.9), respectively, with a ratio (geometric LS mean [90% CI]) of 103 (66.6-158). For participants with severe HI and their healthy matches: AUC₀₋ₜ (h×ng/ml) mean (CV %) was 6814 (52.7) and 2484 (38.7), respectively, with a ratio (geometric LS mean [90% CI]) of 263 (132-523); AUC_{0-∞} (h×ng/ml) mean (CV %) was 5322 (35.2) and 2556 (37.3), respectively, with a ratio (geometric LS mean [90% CI]) of 211 (111-403); Cₘₐₓ (ng/ml) mean (CV %) was 177.0 (37.0) and 245.0 (50.6), respectively, with a ratio (geometric LS mean [90% CI]) of 75.7 (42.2-136). PK parameters for ripretinib were comparable between participants with mild HI and their healthy matches, whereas ripretinib AUC₀₋ₜ and AUC_{0-∞} were both approximately 100% greater and t½ was approximately 46% longer, with mean t½ of 23 versus 16 hours, in participants with moderate HI when compared to matched healthy participants (Cₘₐₓ and tmax were comparable to matched healthy participants). For severe HI, ripretinib AUC₀₋ₜ was approximately 160% greater, AUC_{0-∞} was approximately 111% greater, Cₘₐₓ was approximately 24% lower and t½ was approximately 71% longer, with mean t½ of 24 versus 14 hours, in participants with severe HI when compared to matched healthy participants (tmax was comparable to matched healthy participants). Since ripretinib AUC_{0-∞} could not be estimated for 1 of the 4 participants with severe HI, AUC₀₋ₜ may better represent the magnitude of effect of severe HI given the small sample size for this group. PK parameters for DP-5439 were as follows: • or participants with mild HI and their healthy matches: AUC₀₋ₜ (h×ng/ml) mean (CV %) was 2803 (60.5) and 2938 (91.9), respectively, with a ratio (geometric LS mean [90% CI]) of 98.5 (47.1-206); AUC_{0-∞} (h×ng/ml) mean (CV %) was 2887 (58.9) and 2999 (90), respectively, with a ratio (geometric LS mean [90% CI]) of 99.6 (48.2-206); and Cₘₐₓ (ng/ml) mean (CV %) was 80.43 (53.2) and 113.6 (62.2), respectively, with a ratio (geometric LS mean [90% CI]) of 71.8 (47.0-110).

For participants with moderate HI and their healthy matches: AUC₀₋ₜ (h×ng/ml) mean (CV %) was 4994 (76.2) and 3836 (67), respectively, with a ratio (geometric LS mean [90% CI]) of 120 (59.1-244); AUC_{0-∞} (h×ng/ml) mean (CV %) was 5258 (77.5) and 3916 (65.6), respectively, with a ratio (geometric LS mean [90% CI]) of 122 (60.6-247); Cₘₐₓ (ng/ml) mean (CV %) was 88.53 (58.8) and 127.8 (54.3), respectively, with a ratio (geometric LS mean [90% CI]) of 68.0 (43.4-107). For participants with severe HI and their healthy matches: AUC₀₋ₜ (h×ng/ml) mean (CV %) was 1755 (43.6) and 4263 (88.8), respectively, with a ratio (geometric LS mean [90% CI]) of 56.0 (17.6-179); AUC_{0-∞} (h×ng/ml) mean (CV %) was 1554 (39.4) and 4313 (87.7), respectively, with a ratio (geometric LS mean [90% CI]) of 49.7 (13.1-189); Cₘₐₓ (ng/mL) mean (CV %) was 27.23 (22.3) and 145.1 (66.1), respectively, with a ratio (geometric LS mean [90% CI]) of 21.6 (10.9-42.9).

PK parameters for DP-5439 were generally comparable in participants with mild HI and their healthy matches with the exception of a slightly lower Cₘₐₓ (approximately 28% lower). DP-5439 AUC₀₋ₜ and AUC_{0-∞} were slightly higher (20% and 22%, respectively) in participants with moderate HI relative to matched healthy participants, while Cₘₐₓ was lower by approximately 32%. DP-5439 tmax was significantly delayed (median tmax of 18 versus 6 hours in matched healthy participants) and t½ was approximately 47% longer, with mean t½ of 25 versus 17 hours, in participants with moderate HI relative to matched healthy participants. The M:P ratios based on AUC and Cₘₐₓ were also decreased by 47% and 41%, respectively, in participants with moderate HI. DP-5439 AUC₀₋ₜ was approximately 44% lower and AUC_{0-∞} was approximately 50% lower in participants with severe HI relative to matched healthy participants, while Cₘₐₓ was lower by approximately 78%. DP-5439 tmax was significantly delayed (median tmax of 24 versus 6 hours in healthy participants) and t½ was approximately 77% longer, with mean t½ of 24 versus 14 hours, in participants with severe HI relative to matched healthy participants. The M:P ratios based on AUC and Cₘₐₓ were also decreased by 82% and 73%, respectively, in participants with severe HI. Since DP-5439 AUC_{0-∞} could not be estimated for 1 of the 4 severe HI participants, AUC₀₋ₜ may better represent the magnitude of effect severe HI given the small sample size for this group. Combined (ripretinib+DP-5439) profiles showed that PK parameters were generally comparable in participants with mild HI relative to matched healthy participants. Ripretinib+DP-5439 AUC₀₋ₜ was higher (by approximately 51% and 37%, respectively), AUC_{0-∞} was higher (by approximately 53% and 14%, respectively), and t½ was longer (by approximately 41% and 59%, respectively) in participants with moderate and severe HI relative to matched healthy participants. Ripretinib+DP-5439 Cₘₐₓ was comparable in participants with moderate HI relative to matched healthy participants but was lower by approximately 45% in participants with severe HI. Since combined AUC_{0-∞} could not be estimated for 1 of the 4 severe HI participants, AUC₀₋ₜ may better represent the magnitude of effect of severe HI given the small sample size for this group.

### Unbound Pharmacokinetics Parameters

Both ripretinib and DP-5439 display high binding to plasma proteins in vitro; therefore, clinical samples were collected to investigate the effect of HI on the unbound fraction (fu) % for both analytes. Distributions of individual mean fu % for both ripretinib and DP-5439 show high variability between participants. The ripretinib mean fu % was ≤0.6% in healthy participants and in participants with hepatic impairment. The DP-5439 fu % was ≤0.5% in healthy participants and 0.2% to 1.4% in participants with hepatic impairment. No clear trend could be established between unbound ripretinib and DP-5439 PK parameters and the degrees of HI. However, the relationship between hepatic function tests and unbound ripretinib and DP-5439 exposure was examined and visual assessment showed that HI, as measured by changes in albumin, ALP, ALT, AST, and bilirubin concentrations, did not appear to have an impact on unbound ripretinib or DP-5439 exposure.

### Conclusions

In conclusion, mild HI did not have impact on the PK of ripretinib or DP-5439. In participants with moderate HI, as defined by Child-Pugh criteria, ripretinib AUC values were higher by approximately 99% when compared to a matched control group of healthy participants, whereas ripretinib+DP-5439 AUCs were higher by approximately 51% to 53%. For participants with severe HI, as defined by Child-Pugh criteria, ripretinib AUC₀₋ₜ was approximately 163% greater and Cₘₐₓ was approximately 24% lower when compared to a match control group of healthy participants, whereas combined ripretinib+DP-5439 AUC₀₋ₜ was higher by approximately 37%.

### Example 11. Physiologically Based Pharmacokinetic Modeling to Inform Ripretinib Dosing Recommendations: Drug Interactions and Hepatic Impairment Populations

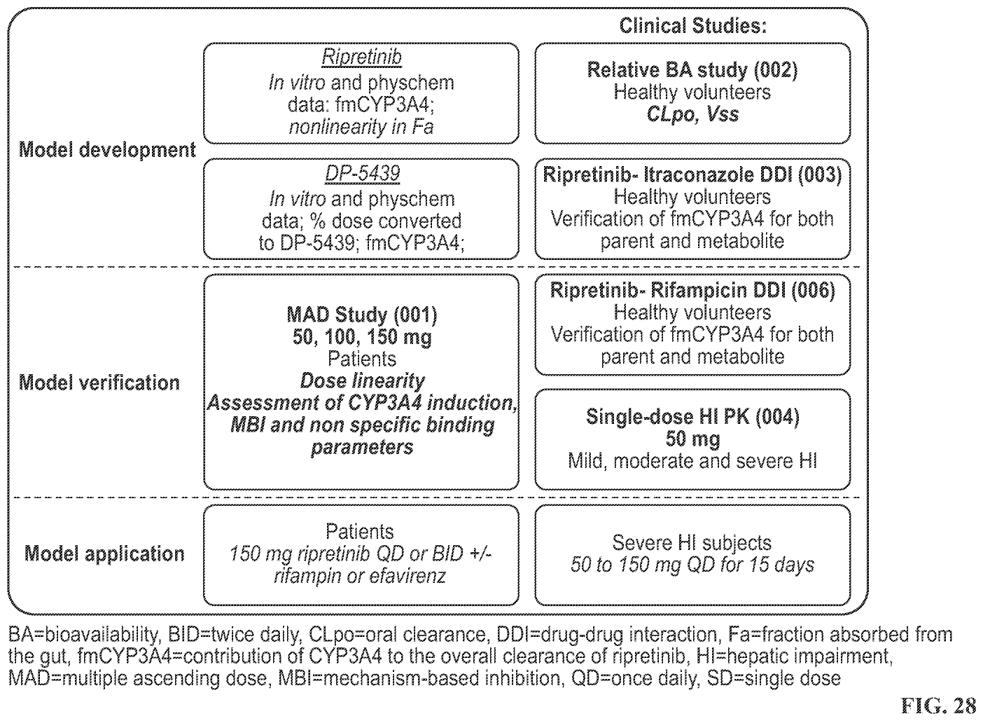

A physiologically based pharmacokinetic (PBPK) model of ripretinib and its active metabolite DP-5439 was used to inform potential ripretinib dose adjustments with strong and moderate cytochrome P450 3A (CYP3A4) inducers and in patients with severe hepatic impairment (HI). In addition, PBPK modeling was used to supplement limited clinical PK data in a severe HI group from a dedicated HI study (n=4 of 8 planned). PBPK modeling provided a good prediction of the HI effect on both parent and metabolite exposure in mild and moderate HI. A ripretinib dose reduction to 50 mg QD led to simulated combined AUC and Cmax ratios of 1.05-fold and 0.84-fold, respectively. A workflow of the PBPK is model is provided in FIG. 28.

### Model Development

A PBPK model that included a mechanistic absorption model was developed. In the base model, the metabolism of ripretinib was assigned to CYP3A4 (59%), CYP2C8 (23%) and CYP2D6 (18%) based on in vitro data. The contribution of CYP3A4 to the overall clearance of ripretinib and DP-5439 was subsequently verified/refined using data from the itraconazole DDI study. The fraction metabolized by CYP3A4 (fmCYP3A4) for ripretinib was set to 54% in the final model. The contribution of CYP3A4 to the overall clearance of DP-5439 was set to 59% in the final model.

### Model Verification

The linked ripretinib and DP-5439 models were further verified using data from the rifampin DDI study.

Following satisfactory recovery of the ripretinib plasma concentration-time profiles after single oral dose to healthy participants, the ripretinib model was applied to assess the recovery of observed PK profiles/exposures of ripretinib following single and repeat (for 14 days) oral doses of 50 to 250 mg QD/twice daily (BID) in virtual participants. Enzyme competitive inhibition constant (Ki) and inactivation rate of enzyme (kinact) values for inhibition of CYP3A4 by ripretinib were incorporated within the PBPK model; CYP3A4 induction data by DP-5439 were also incorporated. Thus, potential auto-induction and auto-inhibition effects were captured within the model.

Single dose (SD) PK data from participants with mild, moderate, and severe HI were used to refine and/or verify the PBPK model. Specifically, the plasma concentration-time profiles of ripretinib and DP-5439 in healthy participants matched to mild (CP-A), moderate (CP-B), and severe (CP-C) HI participants who received a single oral dose of 50 mg in the fasted state were simulated and compared to the observed data. Subsequently, the corresponding plasma concentration-time profiles in HI participants were likewise simulated and compared to the observed data.

### Model Application

Once the ripretinib PBPK model was verified against the available clinical data, predictions of plasma concentrations of ripretinib and DP-5439 in virtual participants following SD and MD administration of 150 mg ripretinib co-administered with the strong CYP3A4 inducer, rifampin, and the moderate CYP3A4 inducer, efavirenz, were generated. The ripretinib PBPK model was also applied to predict changes in drug exposure following MD administration in participants with severe HI.

Predictions of plasma drug concentration-time profiles, clearance and DDIs were performed in the Simcyp Simulator using a population of virtual participants. The default healthy participant population was applied in all simulations involving healthy participants. Default Simcyp parameter values for creating a virtual North European Caucasian population (physiological parameters including hepatic volume and blood flows, enzyme abundances) have been described previously. With the exception of demographic data, all parameter values for the healthy participant population were the same as those used for the North European Caucasian population.

For simulations involving cancer participants, the North European Caucasian population was used to accommodate the wider age range observed in this population (19 to 87 years old; median 61 years). Weibull age distribution was applied with alpha and beta values of 5.47 and 66.5 years for male participants and 5.22 and 68.57 years for female participants. Age, height and weight distributions for the simulated virtual participant population were consistent with those from the first-in-human dose-escalation study in cancer patients.

For simulations involving participants with HI, 3 population models have been developed for the Simcyp Simulator and are based on the 3 Child-Pugh categories (CP-A, CP-B and CP-C). The CYP3A4 abundance values were recently updated to be 107, 70 and 43 pmol P450/mg protein in CP-A, CP-B and CP-C participants, respectively, for Version 21.1 of the Simcyp Simulator (applied in the simulations using Simcyp Version 18.2 in this study). These updated CYP3A4 abundance values were based on recently published literature data, and they were expected to help rectify the trend of over-prediction of HI effects using previous Simcyp versions. When reduced hepatic size was also taken into account, overall CYP3A4 expression in CP-A, CP-B and CP-C participants was reduced to 69%, 36% and 20%, respectively, to that of healthy participants.

### Results

### Model Development

The solubility values of 178 μg/mL in a gastric buffer (pH 2.0) and 123 μg/mL in an intestinal buffer (pH 6.5) were incorporated in the DLM and used as inputs for total segmental solubility for the stomach (178 μg/mL) and all segments of the GI tract (123 μg/mL). Sensitivity analysis of predicted (fa) vs dose was also performed to assess PK nonlinearity. A modest dose-dependent decrease in the fraction absorbed from the gut (fa) was predicted. The predicted fa at 50 mg dose level was 0.90.

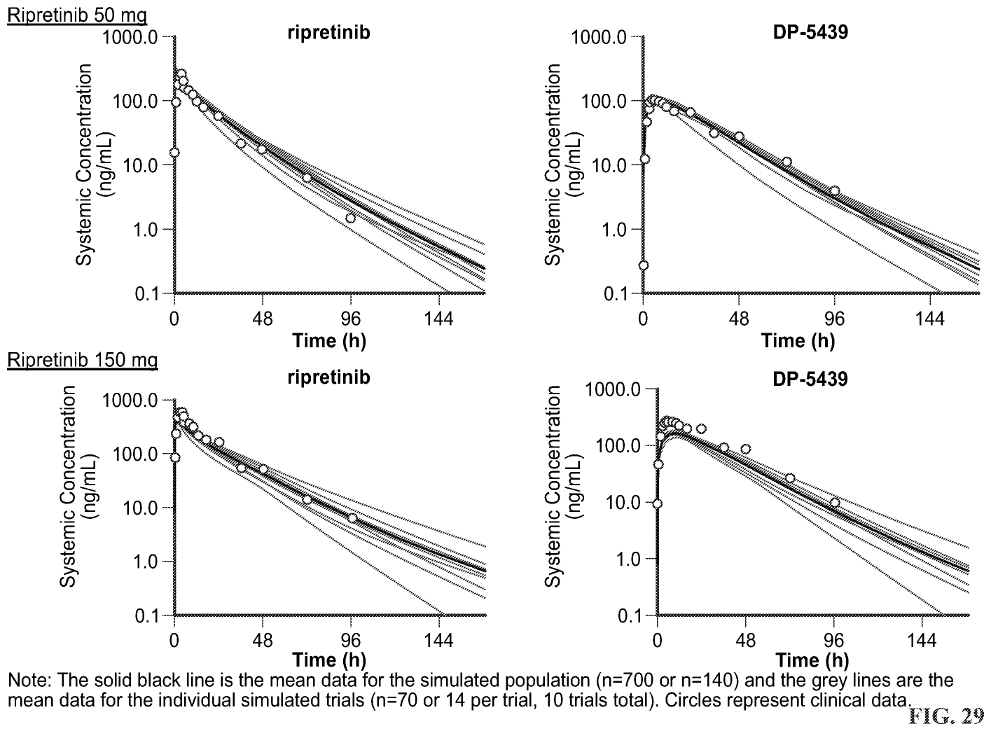

The simulated profiles of ripretinib after a single oral dose (50 mg) of ripretinib in healthy participants using the base model were comparable to the clinical data (FIG. 29). The predicted mean Cₘₐₓ and AUC_{0-∞} values for ripretinib were within 1.13 to 1.14-fold of the observed values. The predicted mean Cₘₐₓ and AUC_{0-∞} values for DP-5439 were within 0.93- to 1.09-fold of the observed values. The simulated profiles of ripretinib after a single oral dose (50 and 150 mg) of ripretinib in healthy participants using the base model were comparable to the clinical data (FIG. 29). The predicted mean Cₘₐₓ and AUC_{0-∞} values for ripretinib were within 0.63- to 0.74-fold of the observed values. The predicted mean Cₘₐₓ and AUC_{0-∞} values for DP-5439 were within 0.57- to 0.67-fold of the observed values. The slight under-prediction of both ripretinib and DP-5439 exposure was likely due to a predicted modest dose-dependent decrease in fa, whereas observed data showed that single-dose ripretinib PK parameters for the 150-mg ripretinib dose appeared to be approximately dose proportional compared to the 50-mg ripretinib dose when given to healthy participants.

Subsequently, the contribution fmCYP3A4 to the formation as well as the clearance of DP-5439 was optimized using data from the itraconazole DDI study. The fmCYP3A4 for ripretinib was reduced from 59% to 54% based on matching the observed itraconazole DDI effects. For the active metabolite DP-5439, it was assumed that CYP3A4 (59%), CYP2C8 (23%) and CYP2D6 (18%) all contributed to the formation of DP-5439 based on the results of sensitivity analysis (results not shown). The contribution of CYP3A4 to the overall clearance of DP-5439 was set to 59%. Simulated and observed plasma concentrations of ripretinib following a single oral dose of ripretinib (50 mg) in the absence of itraconazole and coadministered with itraconazole on the 5th day of 9 days of dosing (200 mg QD) were compared. The simulated profiles of ripretinib and DP-5439 were comparable to the clinical data. Application of the final model showed that the simulated AUC and Cₘₐₓ geometric mean ratios (GMRs) following coadministration of itraconazole with a single dose of 50 mg ripretinib were 2.02 for AUC_{0-∞} and 1.16 for Cₘₐₓ, consistent (within 0.86- to 1.02-fold) with the GMRs of 1.99 and 1.36 for AUC_{0-∞} and Cₘₐₓ, respectively, as observed in a clinical study. The simulated AUC and Cₘₐₓ GMRs of DP-5439 following coadministration of itraconazole with a single dose of 50 mg ripretinib were 1.78 for AUC_{0-∞} and 0.93 for Cₘₐₓ, consistent (within 0.89- to 0.88-fold) with the GMRs of 1.99 and 1.06 for AUC_{0-∞} and Cₘₐₓ, respectively, for observed data.

### Model Verification

Simulated and observed plasma concentrations of ripretinib following a single oral dose of ripretinib (100 mg) in the absence of rifampin and coadministered with rifampin on the 11^{th} day of 15 days of dosing (600 mg QD) were compared. The predicted GMRs for AUC_{0-∞} and Cₘₐₓ of ripretinib following coadministration of rifampin with a single dose of 100 mg ripretinib were 0.32 and 0.65, respectively, compared with the GMRs of 0.39 and 0.82 for AUC_{0-∞} and Cₘₐₓ observed in Clinical Study DCC-2618-01-006. The predicted GMRs for AUC_{0-∞} and Cₘₐₓ of DP-5439 following coadministration of rifampin with a single dose of 100 mg ripretinib were 0.32 and 0.87, respectively, compared with the observed GMRs of 0.43 and 1.37 for AUC_{0-∞} and Cₘₐₓ. Observed DDI data showed an increase in Cₘₐₓ of DP-5439 following rifampin treatment. This may have been partially attributed to the observation that metabolite exposure was generally more variable than parent drug.

CYP3A4 inactivation parameters and induction parameters were subsequently incorporated in the model to assess the net effect on drug clearance at steady state. Due to the uncertainty in the in vitro CYP3A4 MBI data and CYP3A4 induction data, a series of sensitivity analysis was performed. Results showed the effect of changing these parameters on Day 15 drug exposures (150 mg QD and BID) was minimal. Therefore, the in vitro CYP3A4 interaction data were used in the final model. The simulated profiles of ripretinib and DP-5439 in virtual participants were compared to the clinical data. The predicted mean area under the plasma concentration-time curve over 12 hours (AUC₀₋₁₂) and Cₘₐₓ values for ripretinib on Day 1 and Day 15 were within 1.5-fold (majority within 1.25-fold) of the observed values, except for the 250-mg QD dose. The predicted mean AUC₀₋₁₂ values for DP-5439 on Day I were within 2-fold (majority within 1.5-fold) of the observed values, except for the 250-mg QD dose. The mean AUC₀₋₁₂ and Cₘₐₓ values for DP-5439 on Day 15 were under-predicted. A closer examination of the observed data suggested that DP-5439 accumulated more than ripretinib upon MD administration. For example, the reported AUCs for ripretinib and DP-5439 at the 150-mg QD dose level were 1.66 and 5.29, respectively. Following SD administration, DP-5439 had similar t½ (˜17 hours) to ripretinib (˜14 hours) in healthy participants. The mechanisms for the accumulation of DP-5439 were unclear.

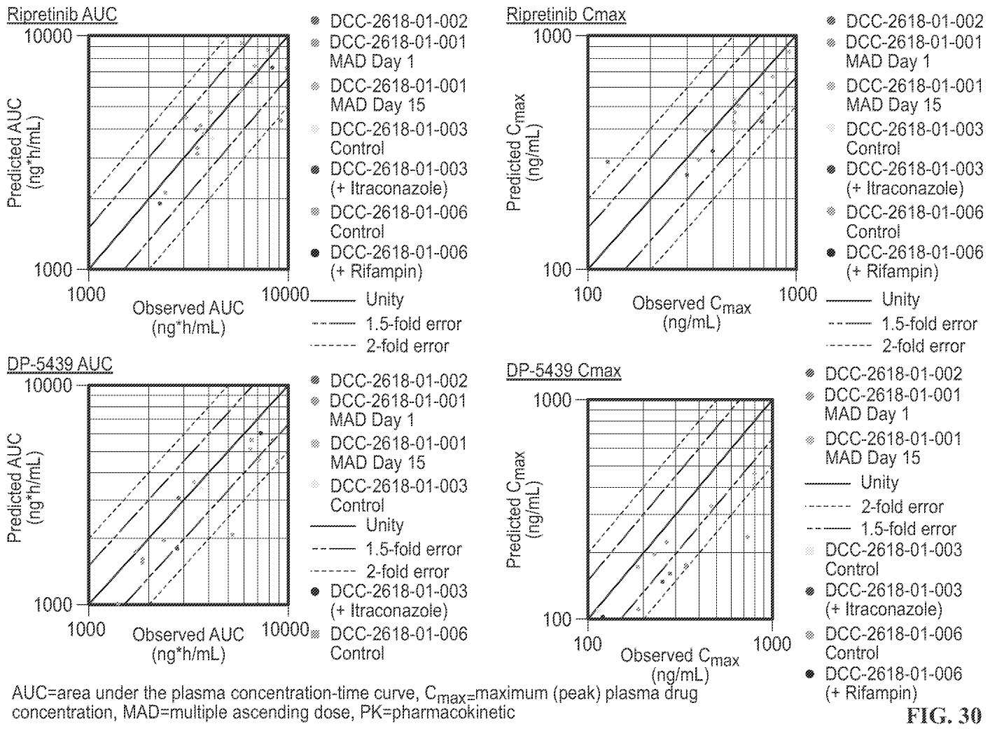

A comparison of the simulated (predicted) and observed PK parameters for all model development and verification results is presented in FIG. 30. Given that the clinically relevant dose of ripretinib is 150 mg QD and the current model performed reasonably well (ie, all predictions fell within 1.25-fold for ripretinib and within 2-fold for DP-5439) at this dose, the model was deemed robust enough to be applied to victim DDI potentials.

Assessment of HI model performance mainly focused on the comparison of the predicted fold-change (HI/healthy) in drug exposures to the observed data in the CP-B group, as negligible PK changes were observed in CP-A group and limited participants (n=4) were enrolled in CP-C group. A comparison of the predicted and observed fold increase in total Cₘₐₓ and AUC_{inf} values of ripretinib and DP-5439 in participants with HI relative to healthy participants who received a single oral dose of 50 mg is shown in Table 9.

The CYP3A4 abundance values in Simcyp Version 21 were implemented in these HI simulations using Version 18.2. These values led to good prediction of the HI effect on both parent and metabolite exposures in the CP-B group. The model predicted a 2.22- and 0.95-fold increase in the AUC_{inf} and Cₘₐₓ of ripretinib in CP-B groups relative to healthy participants, respectively, consistent with the observed fold increase of 1.99- and 1.03-fold, respectively, following the dosing of a single oral dose of 50 mg ripretinib. The model predicted a 1.35- and 0.66-fold increase in the AUC_{inf} and Cₘₐₓ of DP-5439 in CP-B groups relative to healthy participants, respectively, consistent with the observed fold increase of 1.22- and 0.68-fold, respectively, following the dosing of a single oral dose of 50 mg ripretinib. Similarly, the predicted HI effects on both parent and metabolite exposures in the CP-A group were also consistent with the observed data. In general, the predicted HI effects on the total ripretinib AUC_{inf} were higher than that of DP-5439, as HI was expected to reduce the formation of the metabolite (reflected in Cₘₐₓ) as well as the elimination of the metabolite (CYP3A4 enzyme contributed to both the formation and elimination of DP-5439). Overall, the predicted HI effects on total AUC_{inf} following SD administration were small in CP-A and CP-B participants. In CP-C participants, the model predicted a 3.09- and 0.88-fold increase in the AUC_{inf} and Cₘₐₓ of ripretinib relative to healthy participants, respectively, which was higher than the observed fold increase of 2.21- and 0.76-fold, respectively, following the dosing of a single oral dose of 50 mg ripretinib. However, there were limited participants (n=4) enrolled in the CP-C group. The high simulated/observed ratio for DP-5439 in participants with severe HI could have been partly due to the small sample size (n=4) for the CP-C group.

TABLE 9
Comparison of PBPK Simulated vs observed PK parameters for
moderate (CP-B) and severe (CP-C) hepatic impairment groups
receiving single dose ripretinib 50 mg

| CP-B - Moderate HI | | CP-C Severe HI* | |
|---|---|---|---|
| AUC_{0-∞} | Cₘₐₓ | AUC₀₋ₜ | Cₘₐₓ |
| (h · ng/mL) | (ng/mL) | (h · ng/mL) | (ng/mL) |

Ripretinib

| | | | | |
|---|---|---|---|---|
| CP-B/HV Simulated | 2.22 | 0.95 | 2.68 | 0.88 |
| CP-B/HV Observed | 1.99 | 1.03 | 2.63 | 0.76 |
| Simulated/Observed | 1.12 | 0.92 | 1.02 | 1.15 |

DP-5439

| | | | | |
|---|---|---|---|---|
| CP-B/HV Simulated | 1.35 | 0.66 | 1.04 | 0.43 |
| CP-B/HV Observed | 1.22 | 0.68 | 0.56 | 0.22 |
| Simulated/Observed | 1.11 | 0.97 | 1.86 | 1.95 |

AUC₀₋ₜ = area under the plasma concentration-time curve from time zero to time t, AUC_{0-∞} = area under the plasma concentration-time curve from time zero to infinity, Cmax = maximum (peak) plasma drug concentration, CP-B = Child-Pugh moderate HI score, CP-C = Child-Pugh severe HI score, HV = healthy volunteers, PK = pharmacokinetics, PBPK = physiologically based pharmacokinetics.
*Observed and simulated values are based on AUC₀₋ₜ, because AUC_{inf} could not be estimated in 1 out of 4 severe HI participants.

### Model Application

DDI with Strong and Moderate CYP3A4 Inducers

In order to propose dose regimens of ripretinib for patients with GIST taking strong CYP3A4 inducers as comedications, plasma concentrations of ripretinib and DP-5439 following multiple oral doses of ripretinib (150 mg BID or 200 mg BID) coadministered with rifampin on the 11th day of 15 days of dosing (600 mg QD) were simulated and compared to simulated plasma concentrations of ripretinib and DP-5439 following 150 mg QD (standard dose) in the absence of rifampin. Because DP-5439 is a pharmacologically active metabolite, the GMRs for the sum of ripretinib and DP-5439 exposures were also computed. The simulated AUC and Cₘₐₓ GMRs of combined exposure of ripretinib and DP-5439 following 150 mg BID dosing of ripretinib with coadministration of rifampin vs 150-mg QD dosing of ripretinib alone were 0.60 and 0.65, respectively (Table 10). The simulated AUC and Cₘₐₓ GMRs of combined exposure of ripretinib and DP-5439 following 200-mg BID dosing of ripretinib with coadministration of rifampin vs 150-mg QD dosing of ripretinib alone were 0.65 and 0.69, respectively. These results suggest that a ripretinib dose increase from 150 mg QD to 150 mg BID or 200 mg BID in the presence of rifampin does not provide equivalent exposure to 150 mg QD in the absence of rifampin.

To propose dose regimens of ripretinib for patients with GIST taking comedications that are moderate CYP3A4 inducers, plasma concentrations of ripretinib and DP-5439 following multiple oral doses of ripretinib (150 mg BID) coadministered with efavirenz on the 11^{th} day of 15 days of dosing (600 mg QD) were simulated and compared to simulated plasma concentrations of ripretinib and DP-5439 following 150 mg QD (standard dose) in the absence of efavirenz. Predicted mean AUC and Cₘₐₓ values and corresponding GMRs for ripretinib and DP-5439 in the absence and presence of efavirenz are listed in Table 10. The simulated AUC and Cₘₐₓ GMRs of combined exposure of ripretinib and DP-5439 following 150-mg BID dosing of ripretinib with coadministration of efavirenz vs 150 mg QD dosing of ripretinib alone were 0.83 and 0.81, respectively. These results suggest that a ripretinib dose increase from 150 mg QD to 150 mg BID in the presence of efavirenz provided comparable exposure to 150 mg QD in the absence of efavirenz.

TABLE 10
Summary of simulated mean combined PK parameters for once
daily ripretinib in the absence of inducers and twice daily
ripretinib in combination with inducers and associated geometric
mean ratios (90% CI)

| | | | |
|---|---|---|---|
| | | | GMR (BID |
| | Ripretinib | Ripretinib 150 mg | ripretinib + |
| Combined PK | 150 mg | BID + rifampin 600 | inducer/QD |
| Parameter | QD | mg QD | ripretinib alone) |

Ripretinib 150 mg BID + rifampin

| | | | |
|---|---|---|---|
| Cₘₐₓ (ng/mL)* | 1204 | 787 | 0.65 |
| | (90% CI: | (90% CI: | (90% CI: |
| | 1130-1284) | 727-852) | 0.63-0.68) |
| AUC₀₋₂₄ | 19543 | 11778 | 0.60 |
| (h · ng/mL)** | (90% CI: | (90% CI: | (90% CI: |
| | 18093-21109 | 10684-12984) | 0.57-0.64) |

Ripretinib 200 mg BID + rifampin

| | | | |
|---|---|---|---|
| Cₘₐₓ (ng/mL)* | 1204 | 831 | 0.69 |
| | (90% CI; | (90% CI: | (90% CI: |
| | 1130-1284) | 767-901) | 0.66-0.72) |
| AUC₀₋₂₄ | 19543 | 12700 | 0.65 |
| (h · ng/mL)** | (90% CI: | (90% CI: | (90% CI: |
| | 18093-21109) | 11516-14005) | 0.62-0.69) |

Ripretinib 150 mg BID + efavirenz

| | | | |
|---|---|---|---|
| Cₘₐₓ (ng/mL)* | 1204 | 970 | 0.81 |
| | (90% CI: | (90% CI: | (90% CI: |
| | 1130-1284) | 904-1041) | 0.77-0.84) |
| AUC₀₋₂₄ | 19543 | 16249 | 0.83 |
| (h · ng/mL)** | (90% CI: | (90% CI: | (90% CI: |
| | 18093-21109) | 14955-17654) | 0.78-0.88) |

AUC₀₋₂₄ = area under the plasma concentration-time curve over 24 hours, BID = twice daily, CI = confidence interval, Cₘₐₓ = maximum (peak) plasma drug concentration, GMR = geometric mean ratio, QD = once daily.
*Cₘₐₓ (parent) + Cₘₐₓ (metabolite) × (MW(parent)/MW(metabolite).
**AUC₀₋₂₄ (parent) + AUC₀₋₂₄ (metabolite) × (MW(parent)/MW(metabolite) MD simulations in participants with severe HI

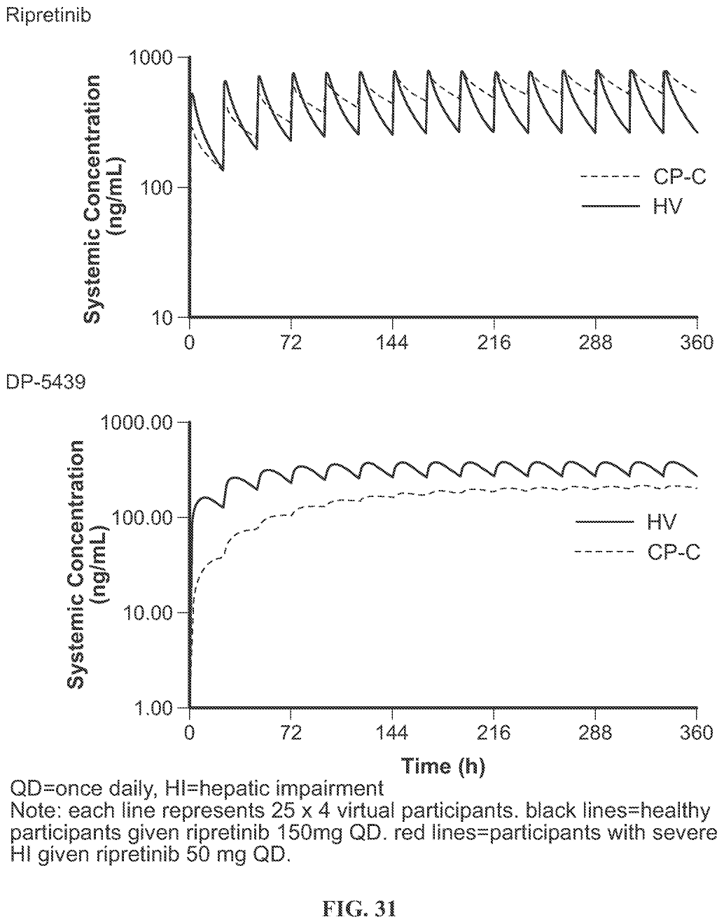

As the predicted HI effects on total AUC_{inf} following SD administration were small in CP-A and CP-B participants, subsequent model application focused on CP-C participants. Simulated total plasma concentration-time profiles of ripretinib in healthy participants who received multiple oral doses of 150 mg ripretinib and participants with severe HI who received multiple oral doses of 50 mg ripretinib for 15 days in the fasted state are shown in FIG. 31.

The simulated AUC and Cₘₐₓ GMRs of combined exposure (after correcting for the molecular weight difference) of ripretinib and DP-5439 following 100-mg QD dosing of ripretinib in CP-C participants vs 150-mg QD dosing of ripretinib in healthy participants were 1.82 and 1.42, respectively (Table 11). The simulated AUC and Cₘₐₓ GMRs of combined exposure of ripretinib and DP-5439 following 50-mg QD dosing of ripretinib in CP-C participants vs 150-mg QD dosing of ripretinib in healthy participants were 1.05 and 0.84, respectively. These results suggest that a ripretinib dose reduction from 150 mg QD to 50 mg QD in CP-C participants provided comparable exposure to 150 mg QD in the healthy participants.

TABLE 11
Summary of predicted mean combined PK parameters for dose-
adjusted ripretinib regimens with severe hepatic impairment (CP-C)
and control group (healthy participants who received ripretinib 150
mg QD) and associated geometric mean ratios (90% CI)

| | Ripretinib + | | | GMR |
|---|---|---|---|---|
| | DP-5439 | | | (Severe HI/ |
| | PK | Severe HI | Control | control |
| Regimen | Parameter | (CP-C) | group | group) |
| Ripretinib | Cₘₐₓ (ng/mL) | 1388 | 978 | 1.42 |
| 100 mg QD | | (90% CI: | (90% CI: | (90% CI: |
| | | 1303-1478) | 928-1031) | 1.31-1.54) |
| | Cₘᵢₙ (ng/mL) | 997 | 414 | 2.41 |
| | | (90% CI: | (90% CI: | (90% CI: |
| | | 926-1074 | 376-455) | 2.12-2.74) |
| | AUC₀₋₂₄ | 27963 | 15368 | 1.82 |
| | (h · ng/mL) | (90% CI: | (90% CI: | (90% CI: |
| | | 26100- | 14352- | 1.65-2.01) |
| | | 29960) | 16456) | |
| Ripretinib | Cₘₐₓ (ng/mL) | 823 | 978 | 0.84 |
| 50 mg QD | | (90% CI: | (90% CI: | (90% CI: |
| | | 775-873) | 928-1031) | 0.78-0.91) |
| | Cₘᵢₙ (ng/mL) | 572 | 414 | 1.38 |
| | | (90% CI: | (90% CI: | (90% CI: |
| | | 532-615) | 376-455) | 1.22-1.57) |
| | AUC₀₋₂₄ | 16180 | 15368 | 1.05 |
| | (h · ng/mL) | (90% CI: | (90% CI: | (90% CI: |
| | | 15135- | 14352- | 0.96-1.16) |
| | | 17296) | 16456) | |

AUC₀₋₂₄ = area under the plasma concentration-time curve over 24 hours, BID = twice daily, CI = confidence interval, Cₘₐₓ = maximum (peak) plasma drug concentration, Cₘᵢₙ = minimum plasma drug concentration, CP-C = Child-Pugh score for severe hepatic impairment, GMR = geometric mean ratio, Control group = healthy participants who received ripretinib 150 mg QD, QD = once daily

### Discussion

In this study, a PBPK model based on prior in vitro and in vivo information on the metabolism and PK of ripretinib was constructed with the aim of predicting plasma concentration-time profiles of ripretinib and its major, pharmacologically active metabolite DP-5439 following single dosing in healthy participants and repeat dosing in cancer patients participant, and to inform the need for dose adjustment of ripretinib in the presence of strong or moderate CYP3A4 inducers and in patients with severe HI.

The key focus of PBPK model development and verification was to estimate the contribution of CYP3A4 to the overall clearance of ripretinib, and the contribution of CYP3A4 to both the formation and clearance of the active metabolite DP-5439. It is important to note that there could have been multiple combinations of the fraction of the dose converted to DP-5439 and contribution of CYP3A4 to the formation of DP-5439 that could have recovered the observed DDI data. This uncertainty cannot be resolved in the absence of human mass balance data.

Subsequently, CYP3A4 inactivation data by ripretinib and CYP3A4 induction data by DP-5439 were incorporated within the PBPK model. Multiple dose simulations (50 to 250 mg QD/BID) were conducted to ensure that the predicted steady-state drug exposures in virtual participants were reasonably consistent with the observed data. Furthermore, the predicted in vivo effect of CYP3A4 inactivation or CYP3A4 induction on hepatic CYP3A4 activity was minimal.

It has been indicated that, for comparisons of predicted vs observed exposure of drugs, within 2-fold of observed data is considered to be “a primary metric for assessment of model fidelity.” However, it should also be noted that, when DDI data are available to optimize the model, this metric is often reduced to within 1.25- or within 1.5-fold. For the PBPK modeling of ripretinib in healthy participants, predictions were considered to be reasonably accurate if the exposures were within 1.5-fold of the observed data.

Model application simulations showed that moderate DDI effects (56% to 68% decrease in AUC₀₋₂₄ of both ripretinib and DP-5439) were predicted with MD administration of ripretinib and concurrent rifampin or efavirenz treatment. Increasing the ripretinib dose from 150 mg QD to 150 mg BID with concomitant use of strong CYP3A4 inducers led to lower combined AUC, compared with 150 mg QD ripretinib alone. Therefore, strong CYP3A4 inducers such as rifampin should be avoided. If strong CYP3A4 inducers cannot be avoided in clinical practice, 150-mg BID regimen may be an option to provide increased exposure for clinical benefit. Per the PBPK model, increasing the ripretinib dose from 150 mg QD to 150 mg BID with concomitant use of moderate CYP3A4 inducers provided comparable exposure (GMR of 0.83 for AUC) to that obtained at 150 mg QD in the absence of efavirenz, supporting this dose adjustment with moderate CYP3A4 inducers.

The PBPK model developed previously was also used to simulate PK of ripretinib and DP-5439 in participants with HI. Assessment of model performance mainly focused on the comparison of the predicted fold-change (HI/healthy) in drug exposures to the observed data in the CP-B group (moderate HI), as negligible PK changes were observed in CP-A group (mild HI) and limited participants (n=4) were enrolled in CP-C group (severe HI).

Applying the updated CYP3A4 abundance values in these HI simulations using Version 18.2 led to good prediction of HI effect on both parent and metabolite exposures in the CP-B group following a single oral dose of 50 mg of ripretinib. The model predicted a 2.22- and 0.95-fold change in the AUC_{inf} and Cₘₐₓ of ripretinib in CP-B groups relative to healthy participants, respectively, consistent with the observed data-fold change of 1.99- and 1.03-fold, respectively. The PBPK model predicted a 1.35- and 0.66-fold change in the AUC_{inf} and Cₘₐₓ of DP-5439 in CP-B groups relative to healthy participants, respectively, consistent with the observed fold change of 1.22- and 0.68-fold, respectively. Similarly, the predicted HI effects on both parent and metabolite exposures in the CP-A group were consistent with the observed data (data not shown). In general, the predicted HI effects on the total ripretinib AUC_{inf} were higher than that of DP-5439, as HI was expected to reduce the formation of the metabolite (reflected in Cₘₐₓ) as well as the elimination of the metabolite (CYP3A4 enzyme contributed to both the formation and elimination of DP-5439). In CP-C participants, the model predicted a 2.68- and 0.88-fold change in the AUC₀₋ₜ and Cₘₐₓ of ripretinib relative to healthy participants, respectively, comparable to the observed changes of 2.63- and 0.76-fold, respectively. However, limited participants (n=4) were enrolled in the CP-C group. In this instance, it has been suggested that PBPK simulations can be used to supplement limited clinical data when recruitment is incomplete for a new molecular entity and can contribute to a totality of evidence. As the predicted HI effects on total AUC_{inf} following SD administration were not considered to be clinically significant with mild and moderate HI, the subsequent model application focused on severe HI. The results showed that a dose reduction from 150 mg to 100 mg QD led to a simulated AUC ratio (CP-C/healthy) and Cₘₐₓ ratio (CP-C/healthy) for the combined total drug exposure of 1.82- and 1.42-fold, respectively. The results showed that a dose reduction from 150 mg to 50 mg QD led to a simulated AUC ratio (CP-C/healthy) and Cₘₐₓ ratio (CP-C/healthy) for the combined total drug exposure of 1.05- and 0.84-fold, respectively. The increased exposure of combined ripretinib plus DP-5439 in patients with severe HI, a potentially vulnerable population, supports dose adjustment to 50 mg QD in such patients.

1-5. (canceled)

6. A method for treating a patient suffering from an advanced gastrointestinal stromal tumor, comprising administering to the patient in need thereof 150 mg once daily of ripretinib, wherein the patient has a pre-existing hepatic impairment.

7. The method of claim 6, wherein the pre-existing hepatic impairment is a pre-existing Child-Pugh class C severe hepatic impairment.
